(19) United States
(12) Patent Application Publication
(10) Pub. No.: US 2013/0273595 A1
(43) Pub. Date: Oct. 17, 2013

(54) METHOD FOR DETERMINING AGE OF GINSENG ROOTS USING CHROMATOGRAMPHY-MASS SPECTROSCOPY

(71) Applicants: KOREA UNIVERSITY RESEARCH AND BUSINE, (US); RURAL DEVELOPMENT ADMINISTRATION, (US)

(72) Inventors: Dongho LEE, Seoul (KR); Nahyun KIM, Seoul (KR); Kemok KIM, Seoul (KR); Jae Won LEE, Seoul (KR); Kyong-Hwan BANG, Suwon (KR); Young-Chang KIM, Suwon (KR); Yoo-Soo SHIN, Seoul (KR); Seon-Woo CHA, Suwon (KR)

(73) Assignees: RURAL DEVELOPMENT ADMINISTRATION, Suwon (KR); KOREA UNIVERSITY RESEARCH AND BUSINESS FOUNDATION, Seoul (KR)

(21) Appl. No.: 13/648,228

(22) Filed: Oct. 9, 2012

Related U.S. Application Data
(63) Continuation of application No. PCT/KR2011/002466, filed on Apr. 11, 2011.

(30) Foreign Application Priority Data
Apr. 9, 2010 (KR) 10-2010-0032980

(51) Int. Cl. G01N 33/487

(52) U.S. Cl. CPC G01N 33/48707; H01J 49/0036
USPC 435/34; 702/19

(57) ABSTRACT

Disclosed is a method for determining the age of ginseng roots using chromatography-mass spectroscopy. It comprises: extracting a metabolome from a ginseng sample; subjecting the metabolome to liquid chromatography-mass spectroscopy (LC/MS) or gas chromatography-mass spectroscopy (GC/MS) to afford an analysis result; converting the LC/MS or GC/MS analysis result to statistically accessible data; and performing a statistical analysis of the data to determine the age of ginseng sample. Based on the metabolite fingerprinting of metabolomics, the method can determine the exact age of ginseng roots from a very small amount of roots within a short time in a non-destructive manner with minimal damage to the roots.

### CROSS REFERENCE TO RELATED APPLICATION

This is a continuation application of International Application No. PCT/KR2011/002466 filed on Apr. 7, 2011, which claims priority to Korean Application No. 10-2010-0032980 filed Apr. 9, 2010, which applications are incorporated herein by reference.

### TECHNICAL FIELD

The present invention relates to the chromatography-mass spectroscopy-based determination of the age of ginseng roots. More particularly, the present invention relates to a method for determining the age of ginseng roots by metabolite fingerprinting analysis using liquid chromatography-mass spectroscopy (LC/MS) or gas chromatography-mass spectroscopy (GC/MS), whereby exact ages of ginseng roots can be rapidly determined and thus reliable systemic distribution management of ginseng products can be constructed.

### BACKGROUND ART

Ginseng is a perennial plant with fleshy roots, belonging to the family Araliaceae. This herb is naturally found in deep mountainous areas and is now artificially cultivated. It is typically about 60 cm tall with a short rhizome stretching upright or slanted. One main trunk stems from the rhizome, with 3-4 verticillate leaves, each consisting of 5 palmate compound leaflets at the end of a long petiole. Small leaves are oval or obovate shaped, tip acuminate, and base narrow and have hairy surface veins with bidentate margins. Ginseng flowers bloom in April and are whitish-green, bunched together in an umbel. Ginseng flowers mature centripetally. Ginseng has a vaguely 5-tooted calyx, 5 stamens, and 5 petals, with 2 pistils. Ginseng berries are round, bunched together in an umbel, and red when mature. Ginseng roots are medicinally used (An Illustrated Guide to Korean Flora, 1993).

In herbal medicine, ginseng is widely used as a medicinal material of an adaptogen for improving stamina and invigorating persons suffering from weakness, weariness, fatigue, inappetence, emesis, and diarrhea. In classic medicinal literature, ginseng is also described to help lung functions, produce vitality, exhibit sedative effects, and enhance renal functions. Reportedly known among the medicinal functions of ginseng are cortical excitation and regulation, balance sensation, anti-fatigue activity, anti-aging activity, immunopotentiation, regulation of cardiac contraction, gonad stimulation, control of hyperglycemia, promotion of protein synthesis, homeostasis maintenance, anticancer activity and detoxification.

Roots of Korean ginseng are fleshy, pale yellowish white, and consist typically of one main root and 2-5 rootlets. The roots are highly apt to bifurcate and change in morphology yearly. Commercially valuable are 4-6-year old roots. In South Korea, red ginseng is made of 6-year-old roots. Each 6-year-old ginseng root is 7-10 cm long, growing maximally up to 34 cm, with a diameter of about 2.5 cm, and weighs about 80 g. Every year, a sprout comes out of the rhizome in soil and the stem and leaves wither and die in autumn.

Most of the ginseng roots that are put on the market are 4˜6 years old. Of them, 6-year-old ginseng roots harvested in autumn are known to have peak medicinal efficacy. Thus, there is a great demand for 6-year-old roots, but their supply is very insufficient, compared to 4- or 5-year-old roots, in practice. In spite of the absolutely insufficient supply of 6-year-old ginseng roots, the market is glutted with them because of fraudulent sales of 4- or 5-year-old roots therefor. Nonetheless, systems for determining the age of ginseng and managing ginseng have not yet been established.

Conventionally, the age of ginseng is determined depending mainly on morphological properties. For example, traces left on the head and rhizome of ginseng roots, the development of rootlets, and overall shapes of roots are analyzed with the naked eye. Alternatively, annual rings are visualized with dye to determine the age of ginseng. Recently, NIR or NMR analysis has been introduced to determine the age of ginseng roots, but is difficult to apply in practice because it is accurate only to a limited degree, and is destructive and requires a long period of time. In full consideration of the current illegal distribution of ginseng, there is a pressing need for exact criteria for determining the age of ginseng whereby systemic distribution management of ginseng can be constructed.

Metabolomics is the scientific study of chemical processes involving compositions and levels of small molecule metabolites (metabolomes) in cells or tissues under various genetic and environmental conditions, using various analysis techniques such as mass spectrometry and NMR analysis, so as to give a more complete picture of living organisms. In metabolomics, metabolic analysis/profiling and deciphering in addition to genomics and proteomics are used to establish more accurate information on organisms.

### SUMMARY OF THE DISCLOSURE

Accordingly, the present invention has been made keeping in mind the above problems occurring in the prior art, and an object of the present invention is to provide an exact and accurate method for determining the age of ginseng roots by analyzing metabolomes on the basis of LC/MS or GC/MS metabolomics, whereby an objective verification system for management of ginseng root products can be established, thereby promising reliable distribution of ginseng product.

It is another object of the present invention to provide a method for determining the age of ginseng roots from a very small amount of roots within a short time in a non-destructive manner with minimal damage to the roots.

Other purposes and advantages of the present invention will be more clearly understood from the following detailed description, claims, and drawings.

In an aspect, the present invention provides a method for determining an age of ginseng roots using chromatography-mass spectroscopy, comprising: extracting a metabolome from a ginseng sample; subjecting the metabolome to liquid chromatography-mass spectroscopy (LC/MS) to afford an analysis result; converting the LC/MS analysis result to statistically accessible data; and performing a statistical analysis of the data to determine the age of ginseng sample.

In another aspect, the present invention provides an apparatus for determining ages of ginseng roots, comprising: a memory for storing standard data of metabolites selected from a ginseng sample, said standard data comprising retention times and molecular weights of the metabolites and being pre-constructed by liquid chromatography-mass spectroscopy; and an analysis means for comparing data measured for a ginseng sample of interest to the standard data, said data measured for the ginseng sample of interest being obtained by liquid chromatography-mass spectroscopy and comprising retention times and molecular weights of the same metabolites as said metabolites.

Based on LC/MS or GC/MS metabolomics, the method and apparatus for determining ages of ginseng roots in accordance with the present invention can perform metabolite profiling for determinants to give information on exact ages of ginseng roots within a short period of time, whereby an objective verification system for the age management of ginseng roots can be established, improving the distribution of ginseng products in terms of reliability.

Also, by utilizing a very small amount of hairy roots, the method and apparatus of the present invention can determine ages of ginseng roots with only minimal damage to the ginseng roots.

Further, it takes a short time, e.g., about 2 hours, for the method and apparatus of the present invention to exactly determining ages of ginseng roots, so that the method and apparatus, based on LC/MS or GC/MS metabolomics, can be very effectively used at the scene.

Other aspects and advantages of the present invention will be described in detail below.

### BRIEF DESCRIPTION OF THE DRAWINGS

The above and other objects, features, and other advantages of the present invention will be more clearly understood from the following detailed description taken in conjunction with the accompanying drawings, in which:

FIG. 1 is a table summarizing data of ginseng taproots and hairy roots used in the present invention;

FIG. 2 is a schematic process flow illustrating the method for determining ages of ginseng roots using LC/MS in accordance with the present invention;

FIGS. 3A and 3B show conditions useful in the LC/MS analysis for determining ginseng root ages;

FIG. 4 is a schematic process flow illustrating the preparation of a sample for use in GC/MS analysis according to the method for determining ages of ginseng roots using GC/MS in accordance with the present invention;

FIG. 5 shows conditions useful in the GC/MS analysis for determining ginseng root ages;

FIGS. 6A˜6C are total ion chromatograms from LC/MS analyses of ginseng taproots (A, B) and hairy roots (C);

FIG. 7 is a PCA plot of total metabolites of ginseng taproots at the age of 1 to 6 years, as constructed by the LC/MS-based method for determining ginseng ages according to the present invention;

FIG. 8 is an HCA plot of total metabolites of ginseng taproots at the age of 1 to 6 years, as constructed by the LC/MS-based method for determining ginseng ages according to the present invention;

FIG. 9 is a PCA plot of total metabolites of ginseng taproots at the age of 4 to 6 years, as constructed by the LC/MS-based method for determining ginseng ages according to the present invention;

FIG. 10 is an HCA plot of total metabolites of ginseng taproots at the age of 4 to 6 years, as constructed by the LC/MS-based method for determining ginseng ages according to the present invention;

FIGS. 11A and 11B are 2D (A) and 3D (B) PCA plots of the metabolites selected by RF, PAM, and PLS-DA from ginseng taproots at the age of 4 to 6 years, as constructed by the LC/MS-based method for determining ginseng ages according to the present invention;

FIG. 12 is an HCA plot of the metabolites selected by RF, PAM, and PLS-DA from ginseng taproots at the age of 4 to 6 years, as constructed by the LC/MS-based method for determining ginseng ages according to the present invention;

FIGS. 13A and 13B are 2D (A) and 3D (B) PCA plots of the metabolites of ginseng hairy roots at the age of 4 to 6 years, as constructed by the LC/MS-based method for determining ginseng ages according to the present invention;

FIG. 14 is an HCA plot of the metabolites of ginseng hairy roots at the age of 4 to 6 years, as constructed by the LC/MS-based method for determining ginseng ages according to the present invention;

FIGS. 15A and 15B are 2D (A) and 3D (B) PCA plots of the metabolites selected by RF, PAM, and PLS-DA from ginseng hairy roots at the age of 4 to 6 years, as constructed by the LC/MS-based method for determining ginseng ages according to the present invention;

FIG. 16 is an HCA plot of the metabolites selected by RF, PAM, and PLS-DA from ginseng hairy roots at the age of 4 to 6 years, as constructed by the LC/MS-based method for determining ginseng ages according to the present invention.

FIGS. 17A and 17B are total ion chromatograms from GC/MS analyses of ginseng taproots (A) and hairy roots (B);

FIG. 18 is a PCA plot of total metabolites of ginseng taproots at the age of 1 to 6 years, as constructed by the GC/MS-based method for determining ginseng ages according to the present invention;

FIG. 19 is an HCA plot of total metabolites of ginseng taproots at the age of 1 to 6 years, as constructed by the GC/MS-based method for determining ginseng ages according to the present invention;

FIG. 20 is a PCA plot of total metabolites of ginseng taproots at the age of 4 to 6 years, as constructed by the GC/MS-based method for determining ginseng ages according to the present invention;

FIG. 21 is an HCA plot of total metabolites of ginseng taproots at the age of 4 to 6 years, as constructed by the GC/MS-based method for determining ginseng ages according to the present invention;

FIGS. 22A and 22B are 2D (A) and 3D (B) PCA plots of the metabolites selected by RF, PAM, and PLS-DA from ginseng taproots at the age of 4 to 6 years, as constructed by the GC/MS-based method for determining ginseng ages according to the present invention;

FIG. 23 is an HCA plot of the metabolites selected by RF, PAM, and PLS-DA from ginseng taproots at the age of 4 to 6 years, as constructed by the GC/MS-based method for determining ginseng ages according to the present invention;

FIGS. 24A and 24B are 2D (A) and 3D (B) PCA plots of the metabolites of ginseng hairy roots at the age of 4 to 6 years, as constructed by the GC/MS-based method for determining ginseng ages according to the present invention;

FIG. 25 is an HCA plot of the metabolites of ginseng hairy roots at the age of 4 to 6 years, as constructed by the GC/MS-based method for determining ginseng ages according to the present invention;

FIGS. 26A and 26B are 2D (A) and 3D (B) PCA plots of the metabolites selected by RF, PAM, and PLS-DA from ginseng hairy roots at the age of 4 to 6 years, as constructed by the GC/MS-based method for determining ginseng ages according to the present invention; and

FIG. 27 is an HCA plot of the metabolites selected by RF, PAM, and PLS-DA from ginseng hairy roots at the age of 4 to 6 years, as constructed by the GC/MS-based method for determining ginseng ages according to the present invention.

### DETAILED DESCRIPTION OF THE DISCLOSURE

In accordance with an aspect thereof, the present invention addresses a method for determining the age of ginseng roots using chromatography-mass spectroscopy, comprising:

1) extracting a metabolome from a ginseng sample;

2) subjecting the metabolome to liquid chromatography-mass spectroscopy (LC/MS) to afford an analysis result;

3) converting the analysis result to statistically accessible data; and

4) performing a statistical analysis of the data to determine the age of ginseng sample.

The method of the present invention, based on chromatography-mass spectroscopy and statistical analysis, can exactly and rapidly determine the age of ginseng roots from even a minimal quantity of ginseng samples.

In step 1), the metabolome for use in metabolomic analysis may be obtained using an extraction method that is well-known in the art. Preferably, it is extracted with 70% MeOH.

To determine the quantity of the metabolome necessary for the LC/MS analysis of step 2), reference may be made to literature. Information about the quantity of extracts, the concentration of analytes, and injection volumes may be established. In addition, two mobile phases that are different in polarity from each other may be employed. To quote an example, a buffer is used for mobile phase A and an organic solvent is used for mobile phase B. Preferably, they have a gradient of concentration according to time. Preferably, example of mobile phase A is water with 0.1% formic acid. Mobile phase B may be a highly polar organic solvent. A non-limiting example of mobile phase B is acetonitrile with 0.1% formic acid. Persons having ordinary skill in the art can choose a suitable organic solvent according to purpose. The flow rate of the mobile phases may range from 200 to 600 μL/min for each column. In one experiment, 500 μL/min was set as a suitable flow rate. In an overall runtime of 12 min, the column may be stabilized by flowing phase B in such a manner that the proportion of phase B is maintained at a rate of 10% for the initial 0.5 min, at a rate of 30% to 2.5 min, at a rate of 60% to 6 min, at a rate of 90% to 9 min, at a rate of 100% to 10.5 min, and then at a rate of 10% to 12 min. The column may be maintained at 35° C.

For high-performance liquid chromatography-mass spectroscopy analysis, components separated on the basis of difference in adsorptivity or partition coefficient between stationary and mobile phases of the analysis column in liquid chromatography are introduced into a mass spectrometer at intervals of retention time. Once a sample is introduced into the mass spectrometer, components of interest are ionized by an ionizing instrument while the mobile phase is removed. In step 2), components of interest may be preferably detected when a reverse phase column is used as the analysis column. The reverse phased column may be a C18 column or a C8 column, with preference for a C18 column. C18 columns guarantee higher resolution and intensity, thus showing improved detection sensitivity. The components separated on the column by liquid chromatography are introduced into a mass spectrometer where they can be ionized using an electrospray ionization machine. In the mass spectrometer, MRM (multiple reaction monitoring) for quantitation aims to improve the signal-to-noise ratio.

Optimal conditions for the negative and positive modes in which mass spectroscopic detection of ginseng metabolomes is performed are established. For example, conditions for the negative mode are as follows: capillary voltage: 2800; cone voltage: 30; collision energy: 6; desolvation temperature: 300° C.; and source temperature: 120° C.

In one embodiment of the present invention, the statistical analysis may be PCA (Principal Component Analysis) or HCA (Hierarchical Cluster Analysis). PCA is a statistical technique designed to convert linearly uncorrelated variables called principal components from possible correlated variables, aiming at the summation and easy analysis of data. That is, PCA allows principal components to be used in subsequent analyses. HCA is a statistical method for finding relatively homogeneous clusters of cases based on measured characteristics. It starts with each case in a separate cluster and then combines the clusters sequentially, reducing the number of clusters at each step until only one cluster is left.

According to one embodiment of the present invention, the ginseng sample comes from a taproot and is used to determine the ages of 1- to 3-year-old ginseng roots. The method of the present invention is advantageous in terms of rapidness and convenience because merely LC/MS analysis data suffices for the exact determination of ages of 1- to 3-year-old ginseng roots.

According to another embodiment of the present invention, the ginseng sample comes from a hairy root (fine root) and is used to determine the ages of 4- to 6-year-old ginseng roots. Merely LC/MS analysis data suffices for exactly determining the ages of 4- to 6-year-old ginseng roots. Like this, the method of the present invention allows even a hairy ginseng root to be a sufficient sample to determine the age of the ginseng with a minimal damage to the ginseng, and its high utility in the market is therefore expected.

In one preferred embodiment of the present invention, the method of the present invention may further comprise executing feature selection to select and analyze an influential and significant metabolite of different ages among the metabolome. The use of a part of the metabolome allows for more exact and rapid determination of the age of ginseng roots.

In another preferred embodiment of the present invention, the feature selection is performed using the three processes of RF (Random Forest, Y. Qiu et al. Metabolomics (2008) 4:337-346), PAM (Prediction Analysis for Microarray, Y. Qiu et al. Metabolomics (2008) 4:337-346), and/or PLS-DA (Partial Least Squares-Discriminant Analysis, Y. Qiu et al. Metabolomics (2008) 4:337-346). In each feature selection process, metabolites are given respective importance scores according to its characteristic algorithm. In RF, for example, individual variables are ranked for significance by importance score. That is, the significance of each metabolite is represented as a numerical value for the influence of the metabolite on the determination of ages of ginseng roots. PAM utilizes the difference between a class centroid and overall centroid for a variable in ranking metabolites. A greater weight is given to a metabolite for which a greater difference between year class mean values and an overall mean value is obtained. For example, respective mean values of metabolite 1 in 3-, 4-, 5-, and 6-year-old roots and in all roots are measured, and the greater the difference between the mean values of the year class and the overall class is, the more significance the metabolite is regarded as having. PLS-DA is a statistical method which uses regression weights in ranking metabolites. In regression modeling, metabolites are assigned respective regression coefficients, and a metabolite with a greater absolute value of its regression coefficient is of more significance. Herein, a regression coefficient is a numerical factor indicative of the influence of a metabolite on the discrimination of the group to which the metabolite belongs.

In one preferred embodiment of the present invention, the ginseng sample is a taproot, and the feature selection is carried out using PLS-DA (Partial Least Squares-Discriminant Analysis) to select and analyze a metabolite whereby the exact ages of 4- to 6-year-old ginseng roots can be determined.

In another preferred embodiment of the present invention, the ginseng sample is a taproot, and the feature selection is carried out using at least two selected from the group consisting of RF (Random Forest), PAM (Prediction Analysis for Microarray) and PLS-DA (Partial Least Squares-Discriminant Analysis) to select and analyze a common metabolite, whereby the exact ages of 4- to 6-year-old ginseng roots can be determined.

In a further preferred embodiment of the present invention, the ginseng sample is a hairy root, and the feature selection is carried out using at least one selected from the group consisting of RF (Random Forest), PAM (Prediction Analysis for Microarray), and PLS-DA (Partial Least Squares-Discriminant Analysis) to select and analyze a metabolite, whereby the exact ages of 4- to 6-year-old ginseng roots can be determined.

In a still further preferred embodiment of the present invention, the ginseng sample is a hairy root, and the feature selection is carried out using at least two selected from the group consisting of RF (Random Forest), PAM (Prediction Analysis for Microarray), and PLS-DA (Partial Least Squares-Discriminant Analysis) to select and analyze a common metabolite, whereby the exact ages of 4- to 6-year-old ginseng roots can be determined.

According to a still further preferable embodiment of the present invention, the metabolite has a retention time (min) and an m/z value of molecular ions of, respectively, 4.1 and 1105, 3.2 and 1436, 4.4 and 971, 2.2 and 499, 3.6 and 883, 4.4 and 971, 4.2 and 841, 4.3 and 1143, or 2.9 and 861 (refer to Table 14). Accordingly, 9 different metabolites can be utilized for determining ages of ginseng roots objectively, exactly, and rapidly.

In accordance with another aspect thereof, the present invention addresses an apparatus for determining ages of ginseng roots, comprising: a memory for storing standard data of metabolites selected from a ginseng sample, said standard data comprising retention times and molecular weights of the metabolites and being pre-constructed by liquid chromatography-mass spectroscopy; and an analysis means for comparing data measured for a ginseng sample of interest to the standard data, said data measured for the ginseng sample of interest being obtained by liquid chromatography-mass spectroscopy and comprising retention times and molecular weights of the same metabolites as said metabolites. Preferably, the metabolites are influential and significant metabolites of different ages. In this apparatus, even a less number of different metabolites of significance allows for the exact and rapid determination of the age of a ginseng root of interest. Further, the apparatus of the present invention, if portable, makes it possible to determine ages of ginseng roots irrespective of place, for example, at the scene. Preferably, the metabolites useful in the present invention are those given in Table 14.

In accordance with a further aspect thereof, the present invention pertains to a method for determining the age of ginseng roots using chromatography-mass spectroscopy, comprising:

1) extracting a metabolome from a ginseng sample;

2) subjecting the metabolome to gas chromatography-mass spectroscopy (GC/MS) to afford an analysis result;

3) converting the analysis result to statistically accessible data; and

4) performing a statistical analysis of the data to determine the age of ginseng sample.

In step 1), the metabolome for use in GC/MS analysis may be obtained using an extraction method that is well-known in the art. Preferably, it is extracted with CHCl₃: MeOH (1:1).

To determine the quantity of the metabolome necessary for the GC/MS analysis of step 2), reference may be made to the literature. Information about the quantity of extracts, the concentration of analytes, and injection volumes may be established. In the approach to factors which significantly differ from one age of ginseng roots to another, experimental data obtained within a time range of 23 min to 24 min 50 sec, which is relevant to major compounds, is excluded so as to increase the detection ratios of minor compounds. For gas chromatography-mass spectroscopy analysis in step 2), components separated on the basis of difference in adsorptivity or partition coefficient between stationary and mobile phases of a capillary column for gas chromatography are introduced into a mass spectrometer at intervals of retention time. Once a sample is introduced into the mass spectrometer, components of interest are ionized by an ionization machine.

In one embodiment of the present invention, the statistical analysis may be PCA (Principal Component Analysis) or HCA (Hierarchical Cluster Analysis).

According to one embodiment of the present invention, the ginseng sample comes from a taproot and is used to determine the ages of 1- or 5-year-old ginseng roots.

In one preferred embodiment of the present invention, the method of the present invention may further comprise executing feature selection to select and analyze an influential and significant metabolite of different ages among the metabolome.

In another preferred embodiment of the present invention, the ginseng sample is a taproot, and the feature selection is carried out using PLS-DA (Partial Least Squares-Discriminant Analysis) to select and analyze a metabolite, whereby the ages of 4- to 6-year-old ginseng roots can be determined.

In a further preferred embodiment of the present invention, the ginseng sample is a taproot and the feature selection is carried out using RF (Random Forest), PAM (Prediction Analysis for Microarray), and PLS-DA (Partial Least Squares-Discriminant Analysis) to select and analyze a common metabolite, whereby the ages of 4- to 6-year-old ginseng roots can be determined.

In a further preferred embodiment of the present invention, the ginseng sample is a hairy root, and the feature selection is carried out using PLS-DA (Partial Least Squares-Discriminant Analysis) to select and analyze a metabolite, whereby the exact ages of 4- to 6-year-old ginseng roots can be determined.

In a still further preferred embodiment of the present invention, the ginseng sample is a hairy root, and the feature selection is carried out using at least two selected from the group consisting of RF (Random Forest), PAM (Prediction Analysis for Microarray), and PLS-DA (Partial Least Squares-Discriminant Analysis) to select and analyze a common metabolite, whereby the ages of 4- to 6-year-old ginseng roots can be determined.

According to a still further preferable embodiment of the present invention, the metabolite has a retention time (min) and an m/z value of molecular ions of, respectively, 16.4 and 73; 26.0 and 204; 9.5 and 73; 20.8 and 204; 26.5 and 73; 6.2 and 57; 3.4 and 244; 31.8 and 217; 9.5 and 147; 11.3 and 147; 22.4 and 73; 19.0 and 149; 16.6 and 71; 7.5 and 57; 3.7 and 171; 32.0 and 441; 30.5 and 217; 18.3 and 73; 12.7 and 73; 9.8 and 133; or 11.0 and 142 (refer to Table 28). Accordingly, 21 different metabolites can be utilized for determining ages of ginseng roots objectively, exactly, and rapidly.

In accordance with another aspect thereof, the present invention addresses an apparatus for determining ages of ginseng roots, comprising: a memory for storing standard data of metabolites selected from a ginseng sample, said standard data comprising retention times and molecular weights of the metabolites, and being pre-constructed by gas chromatography-mass spectroscopy; and an analysis means for comparing data measured for a ginseng sample of interest to the standard data, said data measured for the ginseng sample of interest being obtained by gas chromatography-mass spectroscopy and comprising retention times and molecular weights of the same metabolites as said metabolites. Preferably, the metabolites are influential and significant metabolites of different ages. In this apparatus, even a less number of different metabolites of significance allow for the exact and rapid determination of the age of a ginseng root of interest. Further, the apparatus of the present invention, if portable, makes it possible to determine ages of ginseng roots irrespective of place, for example, at the scene. Preferably, the metabolites useful in the present invention are those given in Table 28.

For use in scientifically determining the exact ages of ginseng roots with minimal damage to the ginseng roots, metabolomes are extracted from ginseng taproots and hairy roots and analyzed by LC/MS or GC/MS under optimal analysis conditions, optionally followed by feature selection. Statistical analysis performed on the data obtained above showed the following results:

1) LC/MS data about taproots suffice for determining ages of 1- to 3-year-old roots exactly, but further requires a feature selection process so as to determine exact ages of 4- to 6-year-old ginseng roots;

2) LC/MS data about hairy roots allows for the determination of exact ages of 4- to 6-year-old ginseng roots without a feature selection process;

3) GC/MS data about taproots requires a feature selection process so as to determine exact ages of 4- to 6-year-old ginseng roots; and

4) GC/MS data about hairy roots requires a feature selection process so as to determine exact ages of 4- to 6-year-old ginseng roots.

### EXAMPLES

A better understanding of the present invention may be obtained through the following examples which are set forth to illustrate, but are not to be construed as limiting, the present invention.

### Example 1

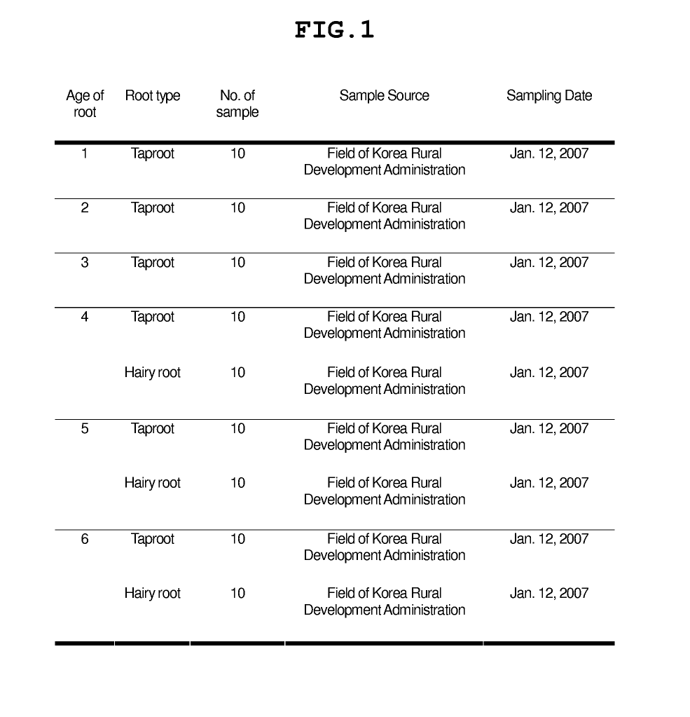

### Preparation of Ginseng Samples (FIG. 1)

From Panax ginseng C. A. Meyer cultivated at the Rural Development Administration, located in Suwon, Korea, 10 taproots of each of 1- to 6-year-old ginseng, and 10 hairy roots of each of 4- to 6-year-old ginseng were harvested on Jan. 12, 2007.

### Example 2

### Preparation of Specimens for LC/MS Analysis

LC/MS was performed using UPLC/Q-ToF MS. Specimens, conditions and statistics for LC/MS analysis were as follows.

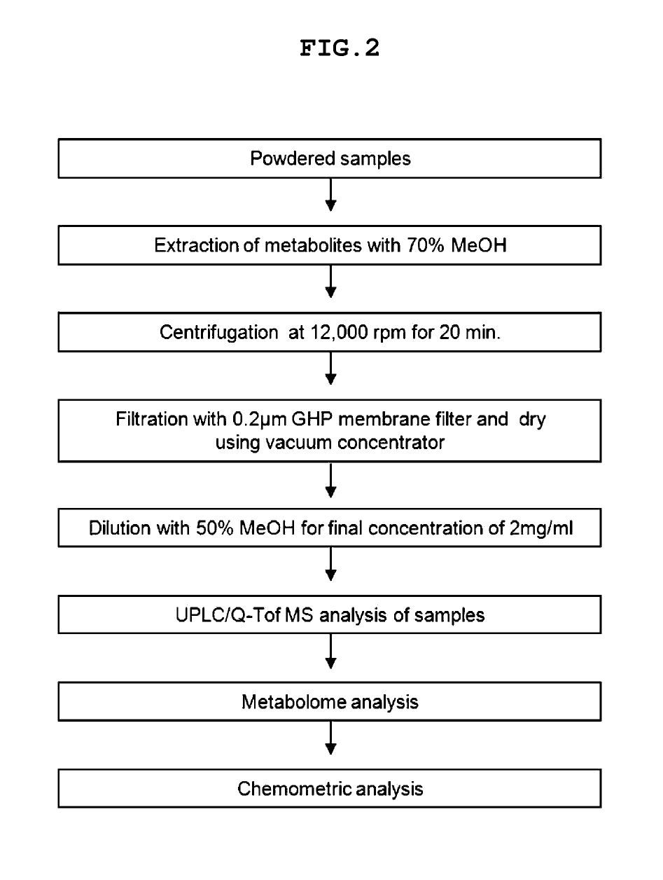

1) Preparation of Specimens for LC/MS Analysis (FIG. 2)

For use in metabolite profiling by LC/MS, metabolites were extracted with 70% aqueous MeOH. To determine the quantity of the metabolites necessary for LC/MS analysis, information about the quantity of extracts, the concentration of analytes, and injection volumes was established by reference to the literature. In this regard, the ginseng samples were cut, freeze-dried just after harvest and powdered, and 50 mg of each powder sample was sonicated for 20 min in 500 μL of 70% aqueous MeOH, followed by centrifugation at 2,000 rpm for 10 min. The supernatant was filtered through a 0.2 μm GHP membrane, and the filtrate was diluted to a final concentration of 2 mg/mL.

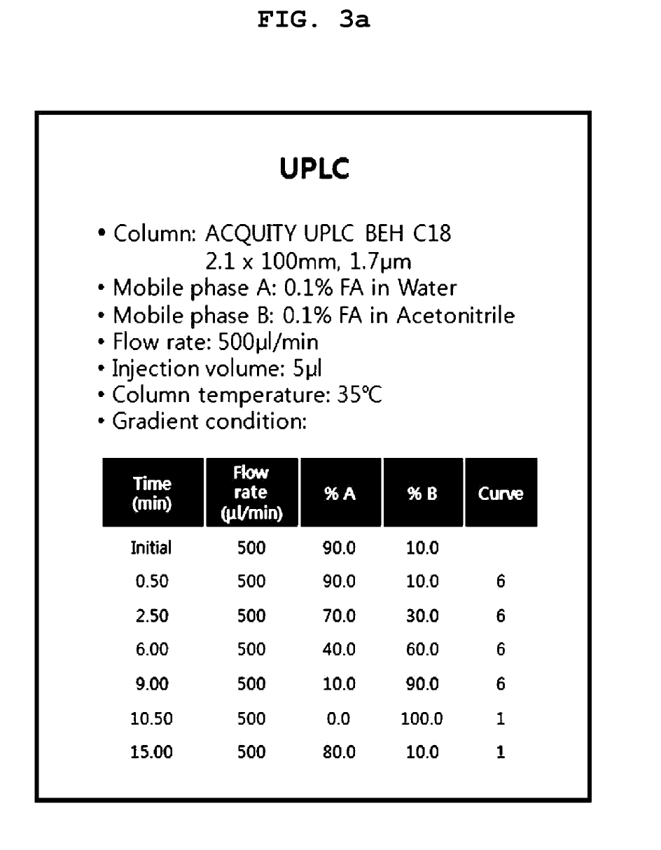

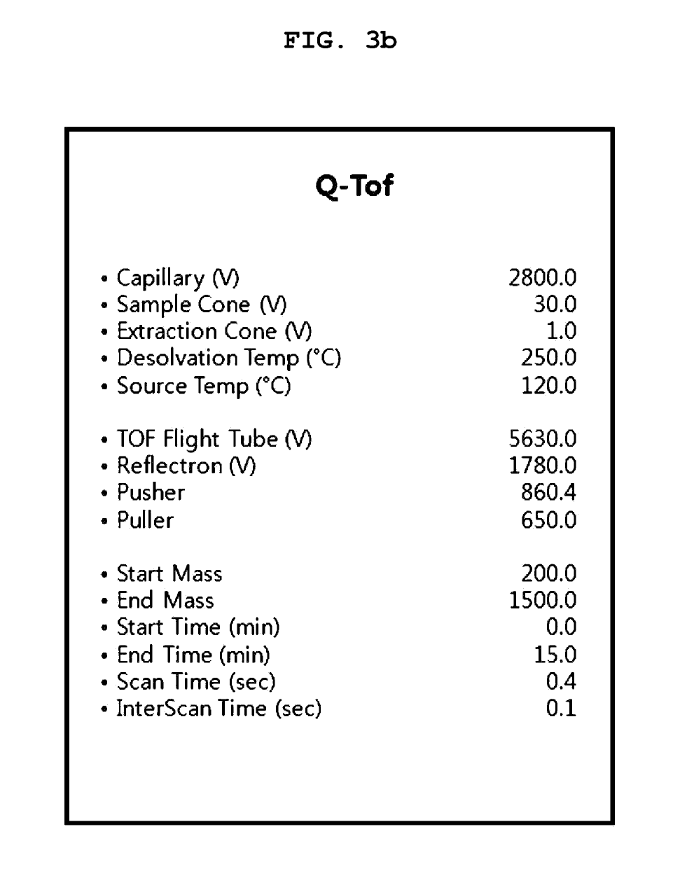

2) LC/MS Conditions (FIGS. 3A and 3B)

(1) UPLC

A Waters ACQUITY UPLC™ system (Waters Corp., MA, U.S.A.) equipped with an ACQUITY UPLC BEH C18 (2.1×100 mm, 1.7) column was utilized. Two mobile phases were used: 0.1% formic acid solution in water (A) and 0.1% formic acid solution in acetonitrile (B). In an overall runtime of 12 min, the column may be stabilized by flowing phase B in such a manner that the proportion of phase B was maintained at a rate of 10% for the initial 0.5 min, at a rate of 30% to 2.5 min, at a rate of 60% to 6 min, at a rate of 90% to 9 min, at a rate of 100% to 10.5 min, and then at a rate of 10% to 12 min. The flow rate, the injection volume, and the column temperature were set to be 500 μL/min, 2 μL, and 35° C., respectively.

(2) Q-ToF MS

Optimal conditions for the negative- and the positive-ion mode in which ginseng metabolites were analyzed by mass spectroscopy using a Q-TOF Micro mass detector (Waters, Manchester, UK) were established. The optimized mass conditions in the negative-ion mode were as follows: capillary voltage=2800 V, cone voltage=30 V, collision energy=6 Ev, desolvation temperature=300° C., and source temperature=120° C.

3) Statistics

With the raw LC/MS data and the data obtained after feature selection, principal component analysis (PCA) and hierarchical cluster analysis (HCA) were performed using MarkerLynx XS (Waters, Manchester, UK) and R version 2.6.1 (R Foundation for Statistical Computing, Vienna, Austria). The LC/MS analysis result such as peaks in a sample were calculated on the basis of RT and m/z data of each peak and normalized by using the MarkerLynx XS application Manager, to be converted to statistically accessible data.

Feature selection, also known as variable selection, is a technique of selecting a subset of relevant features (variables, metabolites) for classification correlation. By removing irrelevant and redundant metabolites which have no significant influence on the determination of ginseng root ages, relevant, influential metabolites are selected for use in determining ginseng root ages.

In the present invention, the three feature selection methods RF (Random Forest), PAM (Prediction Analysis for Microarray), and PLS-DA (Partial Least Squares-Discriminant Analysis) were employed. Importance scores were generated for metabolites by characteristic algorithms of these feature selection methods and were used to select metabolites of significance. In RF, individual metabolites are ranked for significance by importance score. That is, the significance of each metabolite is represented as a numeral value for the influence of the metabolite on the determination of ginseng root ages. PAM utilizes the difference between a class centroid and overall centroid for a variable in ranking metabolites. A greater weight is given to a metabolite for which a greater difference between year class mean values and an overall mean value is obtained. For example, respective mean values of metabolite 1 in 3-, 4-, 5- and 6-year-old roots and in all roots are measured, and the greater the difference between the mean values of the year class and the overall class is, the more significance the metabolite is regarded as having. PLS-DA is a statistical method which uses regression weights in ranking metabolites. In regression modeling, metabolites are assigned respective regression coefficients, and a metabolite with a greater absolute value of its regression coefficient is of more significance. Herein, a regression coefficient is a numeral factor indicative of the influence of a metabolite on the discrimination of the group to which the metabolite belongs.

### Example 3

### Preparation of Specimens for GC/MS Analysis

GC/MS was carried out using a gas chromatography/mass selective detector (GC/MSD). Specimens and conditions for GC/MS analysis were as follows.

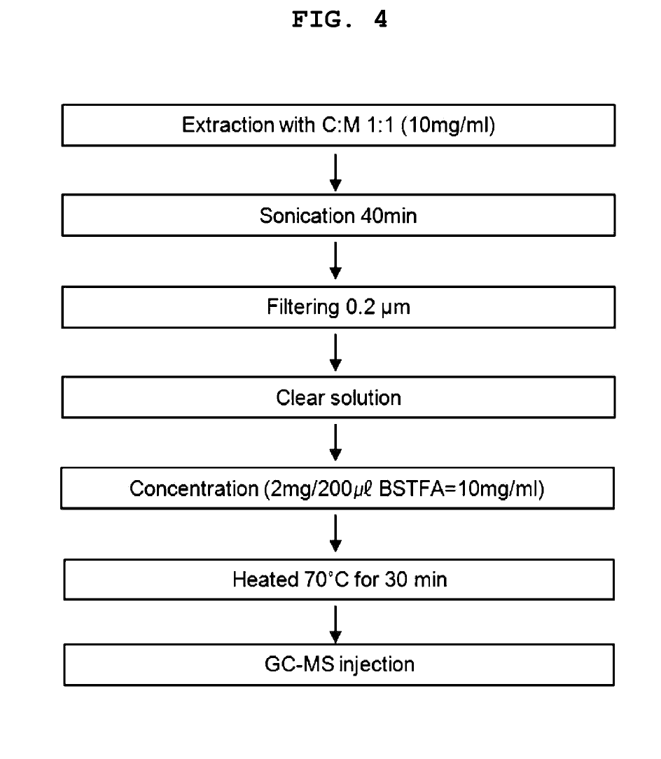

1) Preparation of Specimens for GC/MS Analysis (FIG. 4)

For use in metabolite profiling by GC/MS, metabolites were extracted with CHCl₃: MeOH (1:1). To determine the quantity of the metabolites necessary for GC/MS analysis, information about the quantity of extracts, the concentration of analytes, and injection volumes was established by reference to the literature. Each sample was quantitatively sufficient for conducting experiments therewith in pentaplicate. In this regard, the ginseng samples were cut, freeze-dried just after harvest and powdered, and stored at −80° C. before use. Then, 10 mg of each powder sample was sonicated for 40 min in 1 mL of CHCl₃: MeOH (1:1), followed by centrifugation at 10,000 rpm for 5 min. After 200 μL of the supernatant was concentrated, the concentrate was silylated by reaction with 200 μL of BSTFA for 40 min in a water bath maintained at 70° C.

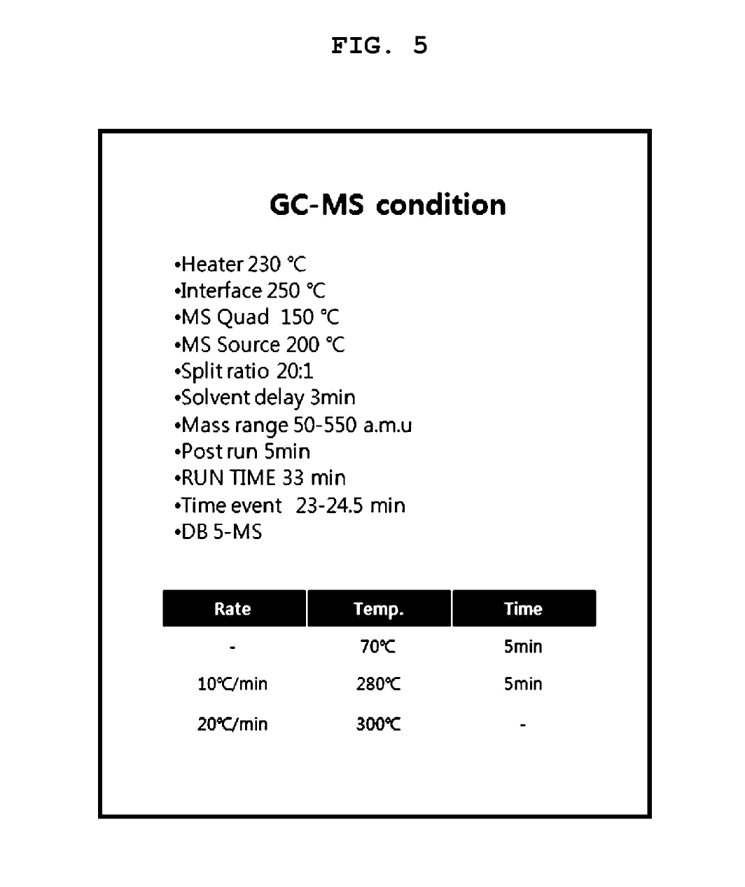

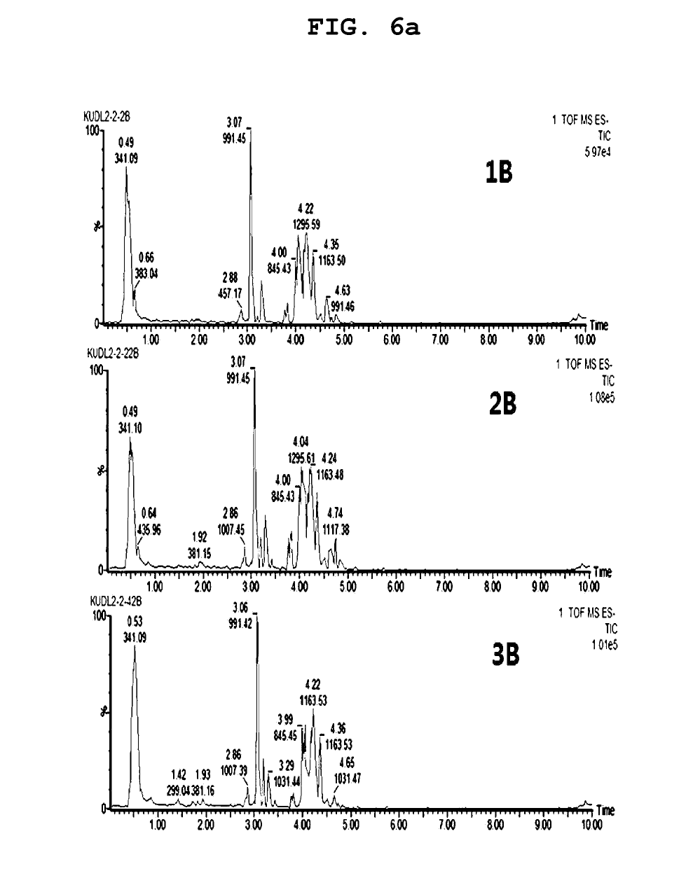

2) GC/MS Conditions (FIG. 5)

For 5 min after a sample was injected, mass values were not detected in order to reduce the solvent load to the instrument. The oven was maintained at 70° C. for 5 min and then heated at a rate of 10° C./min to 280° C. and at a rate of 20° C./min from 280° C. to 300° C. The ion source temperature was 200° C. and injection volume was 1 μL with a split ratio of 20:1. The mass detection range was set to be m/z 50-550. Because the data read in the ion chromatogram for 1 min 50 s between 23 min and 24 min 50 sec after injection corresponded to major compounds which showed relatively high abundance, other compounds had too low abundance values. Accordingly, in this approach to factors which significantly differ from one age of ginseng roots to another, experimental data obtained within a time range of from 23 min to 24 min 50 sec, which was relevant to major compounds, was excluded so as to increase the detection ratios of minor compounds.

3) Statistics

Like the LC/MS data analysis, raw GC/MS data was deconvoluted and assigned using Auto Mass Spectral Deconvolution & Identification System and Spectconnect (http//spectconnect.mit.edu/), and used for feature selection (RF, PAM, and PLS-DA). With the data, PCA (Principal Component Analysis) and HCA (Hierarchical Cluster Analysis) were preformed using MarkerLynx XS (Waters, Manchester, UK) and R version 2.6.1 (R Foundation for Statistical Computing, Vienna, Austria), respectively, for the interpretation of the variations among sample from different ages of ginseng.

### Example 4

### LC/MS Data Analysis for Determination of Ginseng Age

1) LC/MS Analysis Results

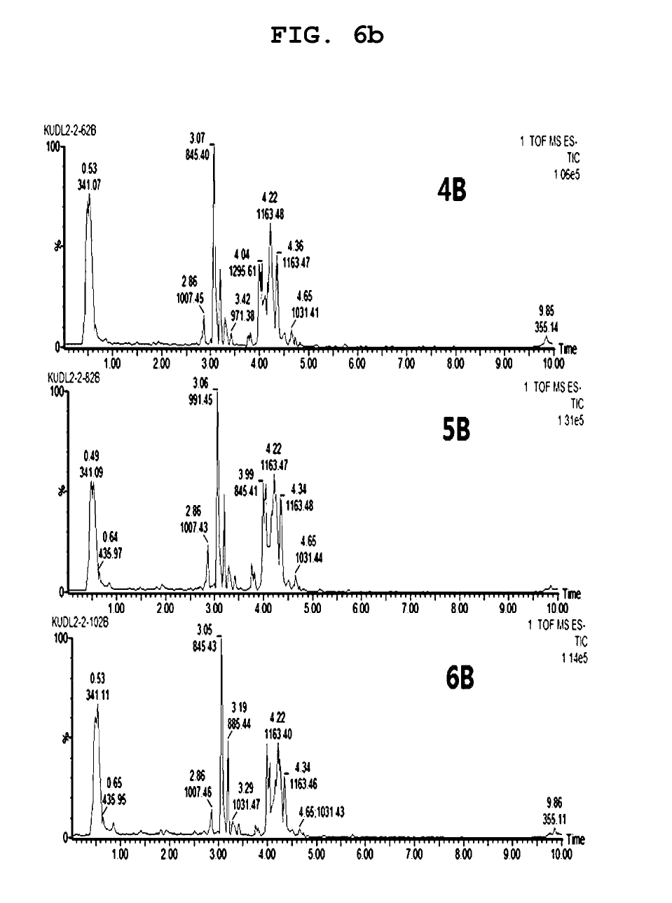

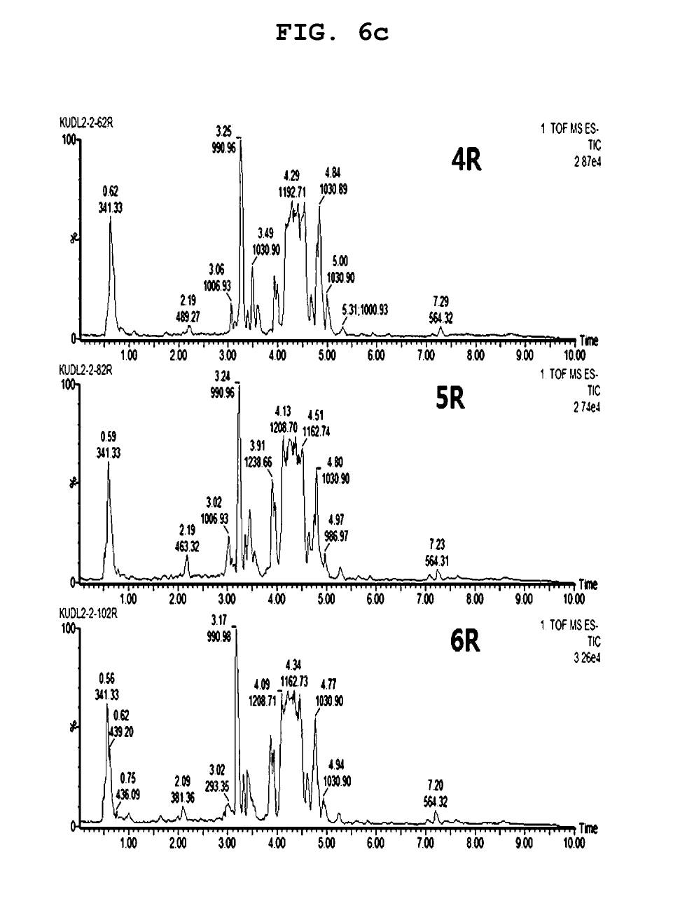

It was difficult to discriminate different ages with the LC/MS data obtained for each of the taproots and hairy roots (FIGS. 6A˜6C).

2) Statics of LC/MS Data (Chemometric Analysis)

With LC/MS data for each of the taproots and the hairy roots, PCA and HCA were preformed. PCA is an unsupervised clustering method, most widely used among multivariate statistical analysis methods, by which a difference between experimental groups can be identified, while HCA is a method by which subjects are classified into clusters and a hierarchy of clusters is built to establish relationships therebetween.

(1) LC/MS Data Analysis for Metabolites of Ginseng Taproot

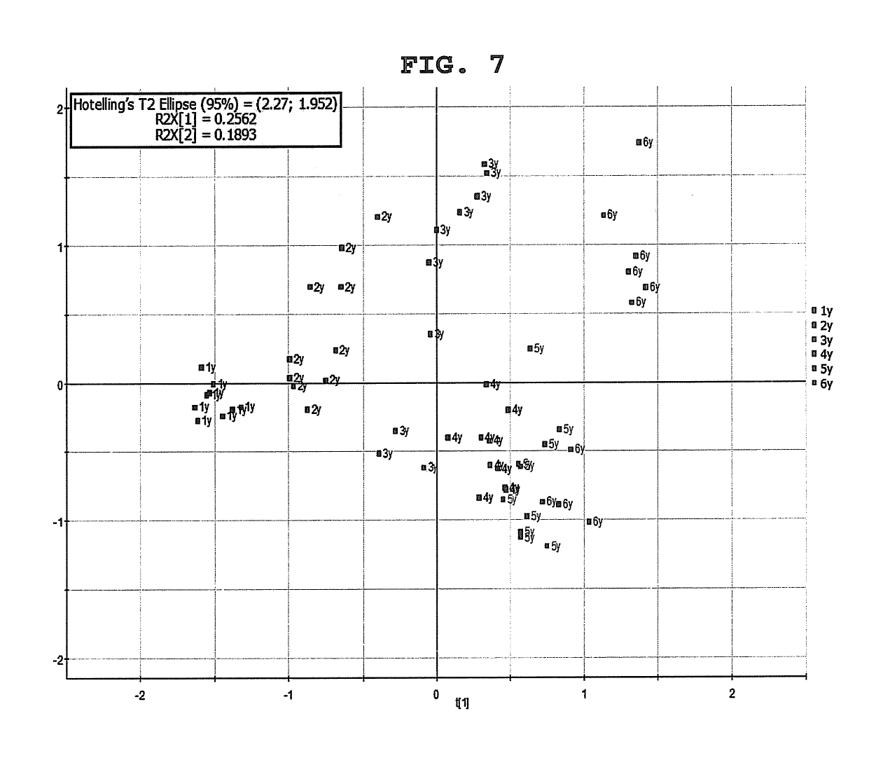

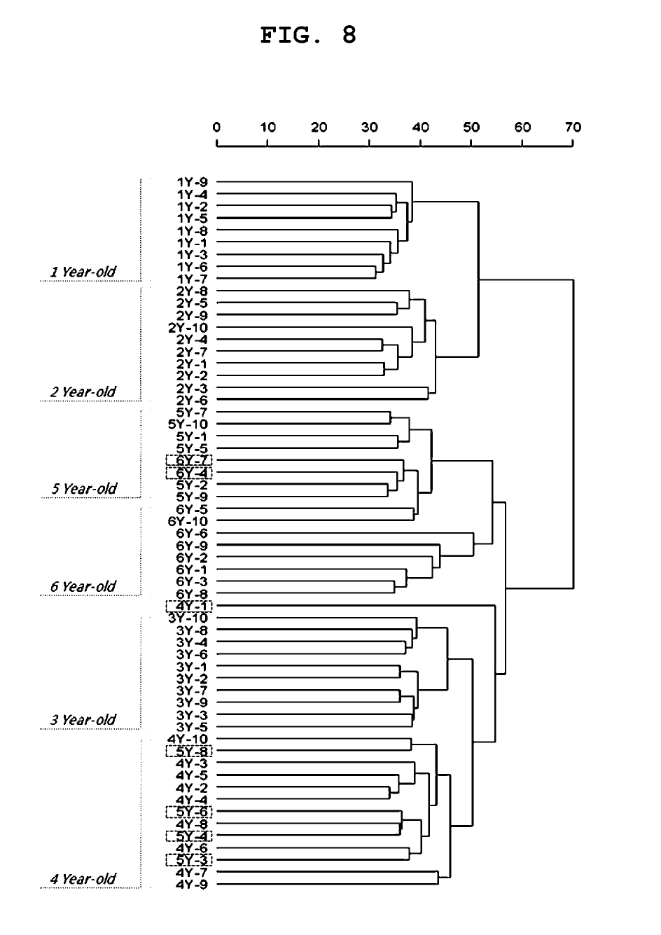

With data about metabolites of each taproot at the age of 1 to 6 years, PCA and HCA were preformed. As a result, merely the raw LC/MS data of ginseng taproots was sufficient to discriminate ginseng roots at the age of 1 to 3 years (FIGS. 7 and 8).

The analysis data for metabolites of each taproot at the age of 1 to 6 years was cross validated for the classification accuracy of each age of ginseng roots using the three feature selection methods RF, PAM, and PLS-DA and was found to classify the ages of ginseng roots with approximately 90% accuracy (Tables 1 to 4). Table 1 summarizes the classification accuracy of taproots at the age of 1 to 6 years determined by RF, PAM, and PLS-DA. Tables 2 to 4 are confusion tables showing the prediction accuracy for ages of the taproots at the age of 1 to 6 years as analyzed by RF, PAM, and PLS-DA, respectively.

TABLE 1

| Classi- | No. of | CV Accuracy (n = 59) | | | | | | |
|---|---|---|---|---|---|---|---|---|
| fication | selected | 1 | 2 | 3 | 4 | 5 | 6 | |
| method | metabolites | Year | Years | Years | Years | Years | Years | Mean |
| RF | 119 | 1.000 | 1.000 | 1.000 | 0.998 | 0.900 | 0.948 | 0.974 |
| PAM | 1146 | 1.000 | 0.900 | 1.000 | 0.900 | 0.796 | 0.966 | 0.926 |
| PLA- | 198 | 1.000 | 1.000 | 1.000 | 1.000 | 0.996 | 1.000 | 0.999 |
| DA | | | | | | | | |

TABLE 2

| | | Predicted Class | | | | | | Prediction |
|---|---|---|---|---|---|---|---|---|
| | | 1 Y | 2 Y | 3 Y | 4 Y | 5 Y | 6 Y | Accuracy |
| True | 1 Y | 450 | 0 | 0 | 0 | 0 | 0 | 1.000 |
| Class | 2 Y | 0 | 500 | 0 | 0 | 0 | 0 | 1.000 |
| | 3 Y | 0 | 0 | 500 | 0 | 0 | 0 | 1.000 |
| | 4 Y | 0 | 0 | 0 | 499 | 1 | 0 | 0.998 |
| | 5 Y | 0 | 0 | 0 | 9 | 450 | 41 | 0.900 |
| | 6 Y | 0 | 0 | 0 | 0 | 26 | 474 | 0.948 |

TABLE 3

| | | Predicted Class | | | | | | Prediction |
|---|---|---|---|---|---|---|---|---|
| | | 1 Y | 2 Y | 3 Y | 4 Y | 5 Y | 6 Y | Accuracy |
| True | 1 Y | 450 | 0 | 0 | 0 | 0 | 0 | 1.000 |
| Class | 2 Y | 50 | 450 | 0 | 0 | 0 | 0 | 0.900 |
| | 3 Y | 0 | 0 | 500 | 0 | 0 | 0 | 1.000 |
| | 4 Y | 0 | 0 | 0 | 450 | 50 | 0 | 0.900 |
| | 5 Y | 0 | 0 | 0 | 0 | 398 | 102 | 0.796 |
| | 6 Y | 0 | 0 | 0 | 0 | 17 | 483 | 0.966 |

TABLE 4

| | | Predicted Class | | | | | | Prediction |
|---|---|---|---|---|---|---|---|---|
| | | 1 Y | 2 Y | 3 Y | 4 Y | 5 Y | 6 Y | Accuracy |
| True | 1 Y | 450 | 0 | 0 | 0 | 0 | 0 | 1.000 |
| Class | 2 Y | 0 | 500 | 0 | 0 | 0 | 0 | 1.000 |
| | 3 Y | 0 | 0 | 500 | 0 | 0 | 0 | 1.000 |
| | 4 Y | 0 | 0 | 0 | 500 | 0 | 0 | 1.000 |
| | 5 Y | 0 | 0 | 2 | 0 | 498 | 0 | 0.996 |
| | 6 Y | 0 | 0 | 0 | 0 | 0 | 500 | 1.000 |

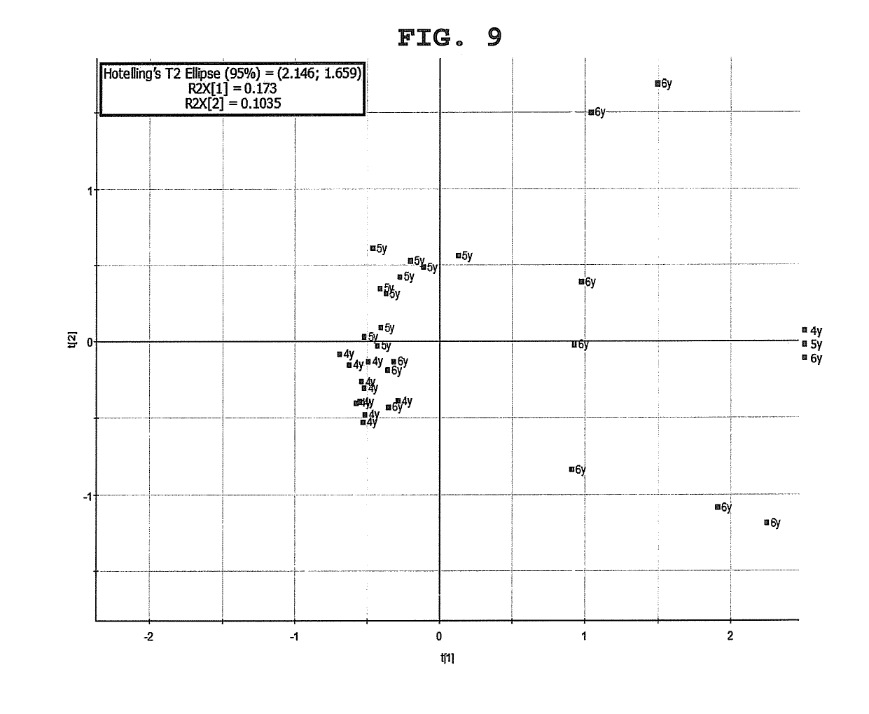

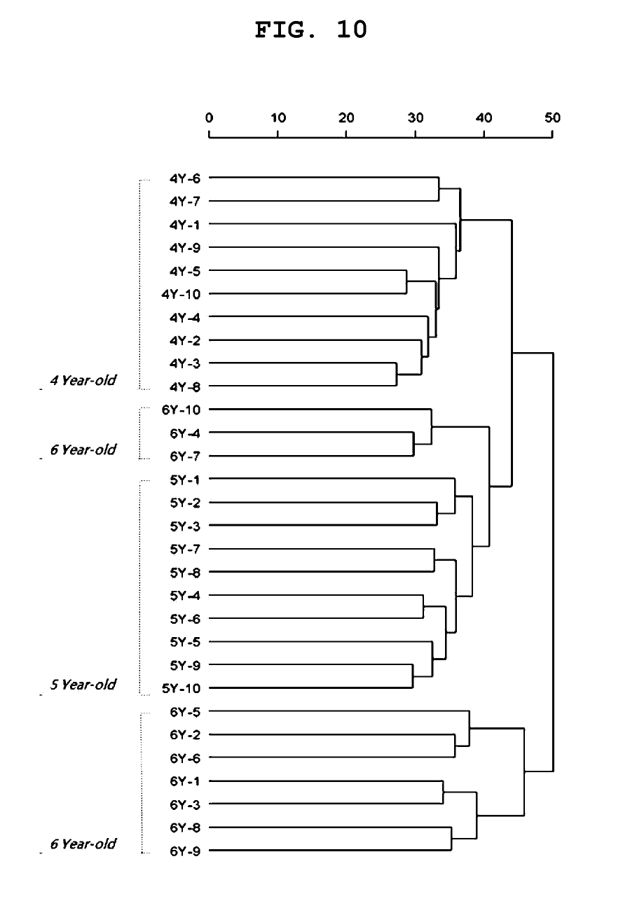

Only with the data of 4- to 6-year-old ginseng roots, which are of main interest to the present invention, the above statistical analysis was preformed. As a result, it was rather difficult to exactly discriminate the ginseng roots at the age of 4 to 6 years with the data of total metabolites (FIGS. 9 and 10).

However, the data of 4- to 6-year-old ginseng taproots were found to allow for the determination of ages of 4- to 6-year-old ginseng roots as analyzed by the three feature selection methods RF, PAM, and PLS-DA, with the perfect discrimination by PLS-DA (Tables 5 to 8). Table 5 summarizes the classification accuracy of taproots at the age of 4 to 6 years determined by RF, PAM, and PLS-DA. Tables 6 to 8 are confusion tables showing the prediction accuracy for ages of the 4- to 6-year-old taproots as analyzed by RF, PAM, and PLS-DA, respectively.

TABLE 5

| Classification | No. of selected | CV Accuracy (n = 30) | | | |
|---|---|---|---|---|---|
| method | metabolites | 4 Years | 5 Years | 6 Years | Mean |
| RF | 73 | 0.995 | 0.804 | 0.974 | 0.924 |
| PAM | 725 | 1.000 | 0.999 | 0.879 | 0.959 |
| PLA-DA | 605 | 1.000 | 1.000 | 1.000 | 1.000 |

TABLE 6

| | | Predicted Class | | | Classification |
|---|---|---|---|---|---|
| | | 4 Y | 5 Y | 6 Y | Accuracy |
| True | 4 Y | 995 | 5 | 0 | 0.995 |
| Class | 5 Y | 125 | 804 | 71 | 0.804 |
| | 6 Y | 3 | 23 | 974 | 0.974 |

TABLE 7

| | | Predicted Class | | | Classification |
|---|---|---|---|---|---|
| | | 4 Y | 5 Y | 6 Y | Accuracy |
| True | 4 Y | 1000 | 0 | 0 | 1.000 |
| Class | 5 Y | 0 | 999 | 1 | 0.999 |
| | 6 Y | 0 | 121 | 879 | 0.879 |

TABLE 8

| | | Predicted Class | | | Classification |
|---|---|---|---|---|---|
| | | 4 Y | 5 Y | 6 Y | Accuracy |
| True | 4 Y | 1000 | 0 | 0 | 1.000 |
| Class | 5 Y | 0 | 1000 | 0 | 1.000 |
| | 6 Y | 0 | 0 | 1000 | 1.000 |

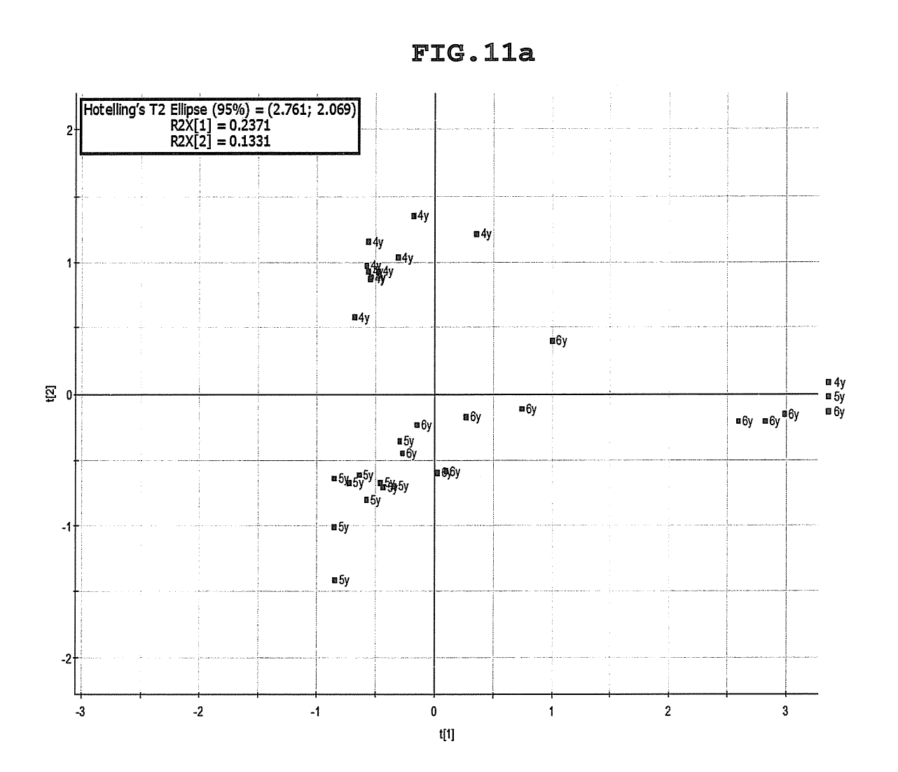

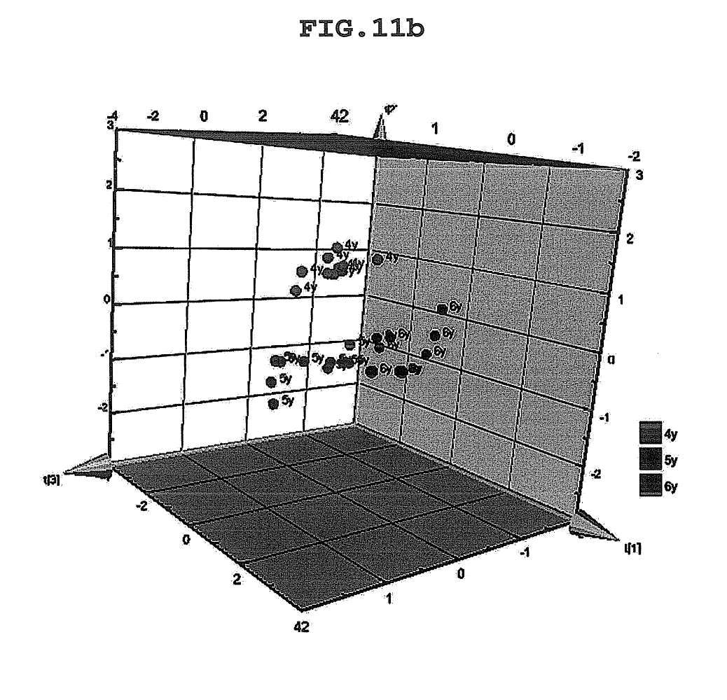

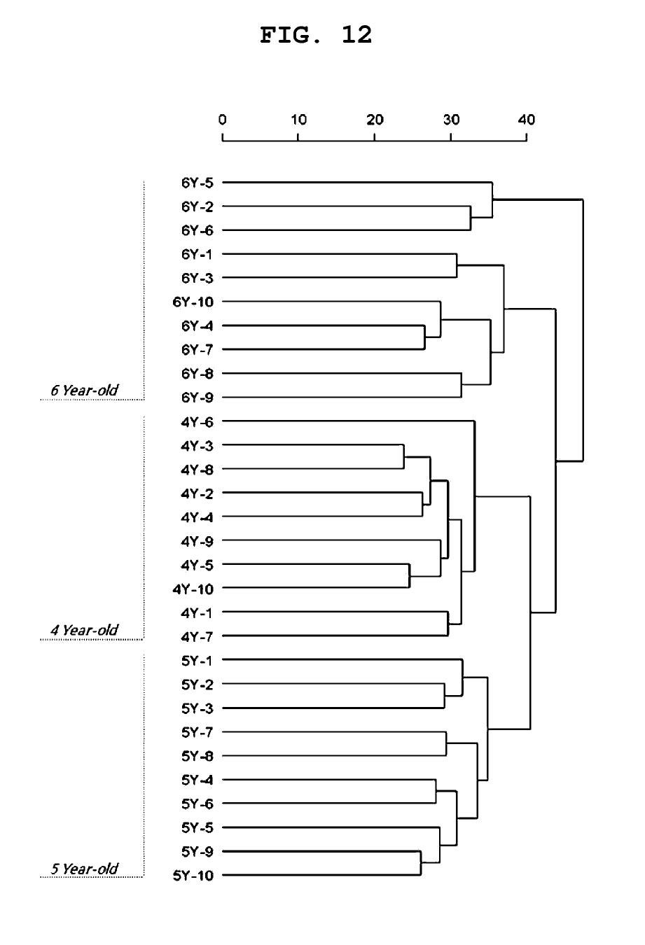

In addition, when evaluated with data of the 606 metabolites of ginseng taproots at the age of 4 to 6 years selected by at least two of RF, PAM, and PLS-DA (Table 9), PCA and HCA were found to determine the exact ages of ginseng roots at the age of 4 to 6 (FIGS. 11A, 11B, and 12).

TABLE 9

| | | Retention | |
|---|---|---|---|
| | | Time | Ion |
| NO | Meta # | (min.) | (m/z) |
| 1 | 358 | 2.7 | 815 |
| 2 | 307 | 3.2 | 695 |
| 3 | 645 | 4.3 | 1074 |
| 4 | 496 | 2.6 | 961 |
| 5 | 811 | 3.0 | 1167 |
| 6 | 694 | 4.0 | 1107 |
| 7 | 823 | 4.0 | 1174 |
| 8 | 815 | 3.0 | 1169 |
| 9 | 224 | 3.0 | 583 |
| 10 | 272 | 3.8 | 642 |
| 11 | 772 | 4.2 | 1143 |
| 12 | 221 | 4.2 | 582 |
| 13 | 539 | 3.3 | 987 |
| 14 | 984 | 3.1 | 1273 |
| 15 | 400 | 4.0 | 845 |
| 16 | 568 | 2.6 | 1007 |
| 17 | 212 | 4.0 | 576 |
| 18 | 237 | 4.0 | 599 |
| 19 | 179 | 4.0 | 553 |
| 20 | 782 | 4.0 | 1153 |
| 21 | 888 | 3.1 | 1223 |
| 22 | 202 | 2.6 | 565 |
| 23 | 1026 | 4.0 | 1296 |
| 24 | 574 | 4.6 | 1015 |
| 25 | 417 | 2.7 | 861 |
| 26 | 654 | 2.9 | 1077 |
| 27 | 509 | 4.3 | 971 |
| 28 | 1068 | 4.2 | 1326 |
| 29 | 1067 | 3.8 | 1326 |
| 30 | 210 | 2.6 | 575 |
| 31 | 102 | 3.4 | 445 |
| 32 | 717 | 3.0 | 1121 |
| 33 | 849 | 4.2 | 1193 |
| 34 | 58 | 2.5 | 323 |
| 35 | 384 | 2.9 | 831 |
| 36 | 475 | 3.2 | 953 |
| 37 | 812 | 3.0 | 1169 |
| 38 | 763 | 4.7 | 1139 |
| 39 | 175 | 2.8 | 549 |
| 40 | 1040 | 3.8 | 1300 |
| 41 | 789 | 4.0 | 1160 |
| 42 | 698 | 4.1 | 1113 |
| 43 | 359 | 4.1 | 815 |
| 44 | 562 | 3.3 | 1005 |
| 45 | 1142 | 3.8 | 1428 |
| 46 | 443 | 4.3 | 901 |
| 47 | 944 | 4.4 | 1252 |
| 48 | 227 | 4.2 | 584 |
| 49 | 154 | 3.5 | 515 |
| 50 | 486 | 5.1 | 957 |
| 51 | 1088 | 3.1 | 1346 |
| 52 | 842 | 4.0 | 1191 |
| 53 | 700 | 4.2 | 1113 |
| 54 | 494 | 2.8 | 961 |
| 55 | 538 | 4.8 | 987 |
| 56 | 890 | 4.0 | 1225 |
| 57 | 124 | 2.1 | 473 |
| 58 | 186 | 3.0 | 560 |
| 59 | 1095 | 3.8 | 1354 |
| 60 | 768 | 3.3 | 1139 |
| 61 | 569 | 3.6 | 1007 |
| 62 | 1148 | 4.3 | 1434 |
| 63 | 580 | 4.3 | 1023 |
| 64 | 265 | 4.1 | 627 |
| 65 | 349 | 4.1 | 805 |
| 66 | 567 | 2.8 | 1007 |
| 67 | 590 | 3.3 | 1031 |
| 68 | 409 | 3.6 | 855 |
| 69 | 470 | 4.6 | 945 |
| 70 | 160 | 2.2 | 531 |
| 71 | 502 | 4.6 | 965 |
| 72 | 226 | 3.0 | 584 |
| 73 | 299 | 4.6 | 679 |
| 74 | 381 | 3.8 | 829 |
| 75 | 907 | 3.9 | 1238 |
| 76 | 455 | 3.3 | 927 |
| 77 | 545 | 4.6 | 991 |
| 78 | 193 | 4.2 | 561 |
| 79 | 610 | 3.0 | 1047 |
| 80 | 259 | 3.8 | 619 |
| 81 | 651 | 4.2 | 1077 |
| 82 | 379 | 2.8 | 829 |
| 83 | 133 | 3.4 | 491 |
| 84 | 738 | 4.2 | 1130 |
| 85 | 50 | 2.6 | 311 |
| 86 | 356 | 4.1 | 815 |
| 87 | 469 | 3.3 | 945 |
| 88 | 690 | 3.8 | 1103 |
| 89 | 551 | 2.8 | 997 |
| 90 | 432 | 3.2 | 885 |
| 91 | 771 | 4.0 | 1143 |
| 92 | 19 | 3.0 | 240 |
| 93 | 526 | 3.0 | 981 |
| 94 | 970 | 4.0 | 1268 |
| 95 | 555 | 5.1 | 1001 |
| 96 | 458 | 2.7 | 929 |
| 97 | 1038 | 3.8 | 1300 |
| 98 | 544 | 3.0 | 991 |
| 99 | 859 | 4.0 | 1201 |
| 100 | 647 | 2.8 | 1075 |
| 101 | 865 | 4.2 | 1210 |
| 102 | 155 | 3.3 | 516 |
| 103 | 1131 | 4.0 | 1405 |
| 104 | 449 | 4.6 | 919 |
| 105 | 1096 | 4.0 | 1354 |
| 106 | 177 | 4.0 | 552 |
| 107 | 586 | 4.7 | 1029 |
| 108 | 291 | 3.8 | 665 |
| 109 | 209 | 2.1 | 575 |
| 110 | 480 | 4.2 | 955 |
| 111 | 345 | 4.0 | 799 |
| 112 | 342 | 2.8 | 795 |
| 113 | 82 | 3.0 | 375 |
| 114 | 683 | 3.3 | 1099 |
| 115 | 125 | 3.4 | 473 |
| 116 | 91 | 2.6 | 405 |
| 117 | 1070 | 3.9 | 1326 |
| 118 | 205 | 4.0 | 571 |
| 119 | 18 | 3.3 | 240 |
| 120 | 86 | 2.7 | 387 |
| 121 | 726 | 4.2 | 1123 |
| 122 | 1069 | 4.0 | 1326 |
| 123 | 401 | 3.1 | 845 |
| 124 | 507 | 3.4 | 971 |
| 125 | 533 | 3.2 | 985 |
| 126 | 781 | 4.0 | 1153 |
| 127 | 554 | 5.3 | 1001 |
| 128 | 505 | 4.7 | 969 |
| 129 | 327 | 4.0 | 773 |
| 130 | 882 | 4.7 | 1217 |
| 131 | 1034 | 3.1 | 1296 |
| 132 | 887 | 3.1 | 1223 |
| 133 | 1018 | 3.8 | 1293 |
| 134 | 515 | 3.8 | 973 |
| 135 | 519 | 4.5 | 975 |
| 136 | 116 | 2.1 | 461 |
| 137 | 407 | 2.7 | 851 |
| 138 | 658 | 4.0 | 1081 |
| 139 | 325 | 4.1 | 769 |
| 140 | 988 | 3.8 | 1276 |
| 141 | 153 | 4.7 | 514 |
| 142 | 912 | 3.8 | 1240 |
| 143 | 999 | 4.4 | 1280 |
| 144 | 262 | 4.3 | 627 |
| 145 | 436 | 4.0 | 889 |
| 146 | 862 | 4.1 | 1209 |
| 147 | 517 | 5.1 | 975 |
| 148 | 184 | 2.6 | 559 |
| 149 | 712 | 4.2 | 1119 |
| 150 | 987 | 4.0 | 1276 |
| 151 | 414 | 5.7 | 861 |
| 152 | 799 | 4.5 | 1163 |
| 153 | 206 | 3.8 | 573 |
| 154 | 218 | 3.6 | 577 |
| 155 | 477 | 3.2 | 953 |
| 156 | 1071 | 3.8 | 1326 |
| 157 | 203 | 2.4 | 567 |
| 158 | 374 | 4.7 | 825 |
| 159 | 1100 | 3.8 | 1358 |
| 160 | 468 | 3.0 | 945 |
| 161 | 445 | 4.0 | 913 |
| 162 | 171 | 4.0 | 540 |
| 163 | 425 | 6.1 | 869 |
| 164 | 1058 | 4.0 | 1324 |
| 165 | 941 | 4.5 | 1252 |
| 166 | 1064 | 4.3 | 1326 |
| 167 | 165 | 3.6 | 531 |
| 168 | 546 | 3.4 | 993 |
| 169 | 503 | 2.8 | 967 |
| 170 | 547 | 3.4 | 993 |
| 171 | 1065 | 4.1 | 1326 |
| 172 | 122 | 2.4 | 473 |
| 173 | 48 | 2.7 | 305 |
| 174 | 1008 | 3.8 | 1286 |
| 175 | 1011 | 4.2 | 1288 |
| 176 | 335 | 4.1 | 789 |
| 177 | 751 | 4.2 | 1135 |
| 178 | 24 | 4.8 | 240 |
| 179 | 973 | 4.2 | 1269 |
| 180 | 324 | 4.1 | 769 |
| 181 | 332 | 4.2 | 783 |
| 182 | 774 | 4.2 | 1149 |
| 183 | 225 | 4.2 | 584 |
| 184 | 378 | 4.2 | 829 |
| 185 | 411 | 3.2 | 859 |
| 186 | 845 | 4.1 | 1193 |
| 187 | 846 | 4.2 | 1193 |
| 188 | 390 | 4.2 | 841 |
| 189 | 20 | 3.4 | 240 |
| 190 | 464 | 4.4 | 939 |
| 191 | 688 | 4.2 | 1101 |
| 192 | 194 | 4.3 | 561 |
| 193 | 948 | 4.2 | 1255 |
| 194 | 382 | 4.2 | 829 |
| 195 | 329 | 3.4 | 781 |
| 196 | 352 | 4.3 | 811 |
| 197 | 88 | 2.5 | 395 |
| 198 | 693 | 4.0 | 1107 |
| 199 | 977 | 4.1 | 1270 |
| 200 | 271 | 4.0 | 642 |
| 201 | 399 | 3.2 | 845 |
| 202 | 94 | 6.2 | 413 |
| 203 | 863 | 4.4 | 1210 |
| 204 | 959 | 3.8 | 1260 |
| 205 | 1017 | 4.0 | 1293 |
| 206 | 465 | 4.4 | 941 |
| 207 | 985 | 3.1 | 1273 |
| 208 | 270 | 3.8 | 637 |
| 209 | 1084 | 3.8 | 1342 |
| 210 | 518 | 3.8 | 975 |
| 211 | 49 | 2.2 | 307 |
| 212 | 950 | 4.4 | 1256 |
| 213 | 508 | 3.6 | 971 |
| 214 | 1130 | 4.0 | 1405 |
| 215 | 758 | 4.2 | 1137 |
| 216 | 260 | 4.4 | 620 |
| 217 | 309 | 3.7 | 708 |
| 218 | 398 | 2.5 | 843 |
| 219 | 706 | 4.7 | 1117 |
| 220 | 500 | 2.7 | 963 |
| 221 | 1144 | 3.8 | 1428 |
| 222 | 577 | 3.0 | 1017 |
| 223 | 1133 | 4.0 | 1405 |
| 224 | 444 | 4.0 | 913 |
| 225 | 387 | 3.1 | 835 |
| 226 | 364 | 4.0 | 819 |
| 227 | 892 | 3.3 | 1226 |
| 228 | 344 | 4.0 | 799 |
| 229 | 350 | 4.1 | 805 |
| 230 | 422 | 4.4 | 868 |
| 231 | 614 | 4.2 | 1051 |
| 232 | 921 | 4.3 | 1246 |
| 233 | 780 | 4.0 | 1151 |
| 234 | 366 | 4.4 | 823 |
| 235 | 353 | 3.6 | 811 |
| 236 | 523 | 2.8 | 977 |
| 237 | 691 | 4.0 | 1105 |
| 238 | 306 | 3.8 | 693 |
| 239 | 442 | 4.0 | 899 |
| 240 | 199 | 4.0 | 563 |
| 241 | 783 | 4.4 | 1155 |
| 242 | 68 | 2.1 | 341 |
| 243 | 595 | 3.4 | 1031 |
| 244 | 251 | 2.3 | 607 |
| 245 | 714 | 4.3 | 1119 |
| 246 | 837 | 4.1 | 1183 |
| 247 | 855 | 3.1 | 1200 |
| 248 | 110 | 3.0 | 457 |
| 249 | 169 | 4.3 | 538 |
| 250 | 664 | 4.7 | 1091 |
| 251 | 456 | 2.7 | 929 |
| 252 | 1063 | 3.8 | 1326 |
| 253 | 158 | 2.2 | 523 |
| 254 | 1036 | 3.1 | 1296 |
| 255 | 1029 | 4.4 | 1296 |
| 256 | 1097 | 3.8 | 1354 |
| 257 | 848 | 4.1 | 1193 |
| 258 | 241 | 3.0 | 603 |
| 259 | 114 | 2.1 | 459 |
| 260 | 866 | 4.1 | 1210 |
| 261 | 128 | 2.9 | 487 |
| 262 | 1031 | 4.0 | 1296 |
| 263 | 1123 | 3.8 | 1394 |
| 264 | 573 | 5.5 | 1015 |
| 265 | 348 | 4.3 | 803 |
| 266 | 363 | 4.3 | 819 |
| 267 | 525 | 4.6 | 981 |
| 268 | 341 | 4.6 | 793 |
| 269 | 362 | 4.0 | 819 |
| 270 | 1124 | 3.8 | 1394 |
| 271 | 81 | 6.0 | 369 |
| 272 | 334 | 3.3 | 783 |
| 273 | 59 | 2.7 | 323 |
| 274 | 104 | 3.4 | 447 |
| 275 | 733 | 4.0 | 1127 |
| 276 | 1137 | 3.0 | 1419 |
| 277 | 560 | 4.8 | 1005 |
| 278 | 340 | 5.7 | 793 |
| 279 | 428 | 3.4 | 883 |
| 280 | 310 | 2.8 | 713 |
| 281 | 540 | 4.6 | 987 |
| 282 | 514 | 3.3 | 973 |
| 283 | 976 | 4.2 | 1270 |
| 284 | 816 | 4.7 | 1170 |
| 285 | 937 | 4.4 | 1251 |
| 286 | 331 | 3.4 | 781 |
| 287 | 40 | 2.7 | 290 |
| 288 | 239 | 2.8 | 601 |
| 289 | 215 | 3.8 | 577 |
| 290 | 10 | 2.2 | 240 |
| 291 | 1000 | 4.2 | 1280 |
| 292 | 684 | 4.8 | 1099 |
| 293 | 570 | 3.4 | 1009 |
| 294 | 579 | 3.3 | 1023 |
| 295 | 1081 | 4.2 | 1340 |
| 296 | 983 | 4.2 | 1273 |
| 297 | 386 | 4.0 | 835 |
| 298 | 1002 | 3.8 | 1282 |
| 299 | 1086 | 3.1 | 1346 |
| 300 | 880 | 4.1 | 1217 |
| 301 | 899 | 4.4 | 1230 |
| 302 | 1152 | 3.8 | 1458 |
| 303 | 372 | 6.1 | 825 |
| 304 | 510 | 3.4 | 971 |
| 305 | 383 | 4.3 | 829 |
| 306 | 600 | 4.4 | 1041 |
| 307 | 990 | 3.8 | 1276 |
| 308 | 454 | 3.4 | 927 |
| 309 | 127 | 7.0 | 476 |
| 310 | 276 | 2.7 | 645 |
| 311 | 28 | 3.2 | 244 |
| 312 | 157 | 3.6 | 517 |
| 313 | 113 | 6.6 | 458 |
| 314 | 42 | 2.9 | 293 |
| 315 | 662 | 4.7 | 1084 |
| 316 | 183 | 3.0 | 559 |
| 317 | 829 | 4.4 | 1178 |
| 318 | 43 | 3.0 | 293 |
| 319 | 484 | 4.4 | 955 |
| 320 | 441 | 4.3 | 897 |
| 321 | 643 | 4.7 | 1073 |
| 322 | 236 | 3.8 | 599 |
| 323 | 993 | 4.1 | 1278 |
| 324 | 531 | 3.2 | 984 |
| 325 | 410 | 2.9 | 857 |
| 326 | 36 | 4.6 | 290 |
| 327 | 972 | 4.0 | 1269 |
| 328 | 112 | 6.3 | 457 |
| 329 | 140 | 4.7 | 493 |
| 330 | 222 | 4.3 | 582 |
| 331 | 318 | 3.6 | 739 |
| 332 | 584 | 3.4 | 1025 |
| 333 | 431 | 4.1 | 885 |
| 334 | 960 | 4.3 | 1262 |
| 335 | 457 | 4.0 | 929 |
| 336 | 773 | 4.2 | 1144 |
| 337 | 208 | 4.1 | 574 |
| 338 | 759 | 4.5 | 1137 |
| 339 | 653 | 4.3 | 1077 |
| 340 | 596 | 3.3 | 1031 |
| 341 | 247 | 2.5 | 605 |
| 342 | 627 | 2.9 | 1060 |
| 343 | 244 | 4.4 | 604 |
| 344 | 920 | 4.1 | 1246 |
| 345 | 396 | 3.4 | 841 |
| 346 | 246 | 3.2 | 605 |
| 347 | 187 | 2.5 | 561 |
| 348 | 275 | 2.2 | 645 |
| 349 | 178 | 2.8 | 553 |
| 350 | 38 | 3.0 | 290 |
| 351 | 231 | 2.5 | 595 |
| 352 | 99 | 2.8 | 443 |
| 353 | 132 | 2.1 | 491 |
| 354 | 269 | 3.4 | 637 |
| 355 | 67 | 2.4 | 341 |
| 356 | 957 | 3.8 | 1259 |
| 357 | 268 | 3.8 | 637 |
| 358 | 852 | 4.4 | 1195 |
| 359 | 689 | 3.8 | 1103 |
| 360 | 235 | 4.3 | 597 |
| 361 | 832 | 4.4 | 1179 |
| 362 | 1106 | 3.1 | 1369 |
| 363 | 172 | 6.5 | 545 |
| 364 | 867 | 4.0 | 1210 |
| 365 | 238 | 2.8 | 601 |
| 366 | 501 | 3.0 | 965 |
| 367 | 594 | 4.6 | 1031 |
| 368 | 493 | 5.1 | 961 |
| 369 | 182 | 2.1 | 557 |
| 370 | 640 | 3.4 | 1072 |
| 371 | 711 | 4.5 | 1119 |
| 372 | 214 | 3.8 | 577 |
| 373 | 1145 | 4.3 | 1434 |
| 374 | 216 | 2.8 | 577 |
| 375 | 301 | 2.5 | 683 |
| 376 | 735 | 3.3 | 1129 |
| 377 | 729 | 4.0 | 1125 |
| 378 | 111 | 2.1 | 457 |
| 379 | 625 | 4.6 | 1059 |
| 380 | 191 | 4.4 | 561 |
| 381 | 666 | 4.7 | 1091 |
| 382 | 375 | 3.8 | 827 |
| 383 | 430 | 4.1 | 885 |
| 384 | 678 | 4.2 | 1097 |
| 385 | 728 | 2.9 | 1124 |
| 386 | 482 | 4.2 | 955 |
| 387 | 242 | 4.2 | 604 |
| 388 | 979 | 4.2 | 1270 |
| 389 | 856 | 4.1 | 1201 |
| 390 | 485 | 5.2 | 957 |
| 391 | 189 | 4.2 | 561 |
| 392 | 207 | 4.2 | 574 |
| 393 | 650 | 4.2 | 1077 |
| 394 | 720 | 4.2 | 1123 |
| 395 | 1023 | 4.2 | 1295 |
| 396 | 796 | 4.2 | 1163 |
| 397 | 346 | 4.4 | 803 |
| 398 | 929 | 4.3 | 1249 |
| 399 | 857 | 4.0 | 1201 |
| 400 | 377 | 4.3 | 829 |
| 401 | 365 | 4.4 | 823 |
| 402 | 479 | 4.3 | 955 |
| 403 | 351 | 3.6 | 811 |
| 404 | 450 | 4.4 | 925 |
| 405 | 889 | 4.4 | 1223 |
| 406 | 566 | 3.6 | 1007 |
| 407 | 245 | 4.2 | 604 |
| 408 | 367 | 3.9 | 823 |
| 409 | 996 | 4.3 | 1279 |
| 410 | 1103 | 4.2 | 1364 |
| 411 | 675 | 4.0 | 1094 |
| 412 | 919 | 4.1 | 1246 |
| 413 | 975 | 4.4 | 1269 |
| 414 | 810 | 3.4 | 1167 |
| 415 | 161 | 3.6 | 531 |
| 416 | 989 | 4.0 | 1276 |
| 417 | 121 | 3.3 | 472 |
| 418 | 96 | 2.2 | 415 |
| 419 | 911 | 4.0 | 1240 |
| 420 | 511 | 3.3 | 973 |
| 421 | 806 | 4.1 | 1167 |
| 422 | 248 | 3.8 | 606 |
| 423 | 672 | 2.6 | 1093 |
| 424 | 419 | 4.0 | 862 |
| 425 | 151 | 3.2 | 513 |
| 426 | 285 | 2.6 | 653 |
| 427 | 1098 | 3.7 | 1356 |
| 428 | 16 | 2.8 | 240 |
| 429 | 217 | 2.8 | 577 |
| 430 | 321 | 2.8 | 740 |
| 431 | 333 | 4.2 | 783 |
| 432 | 1140 | 3.8 | 1426 |
| 433 | 743 | 3.3 | 1131 |
| 434 | 549 | 2.8 | 997 |
| 435 | 1061 | 3.8 | 1324 |
| 436 | 513 | 3.0 | 973 |
| 437 | 677 | 4.0 | 1095 |
| 438 | 146 | 2.9 | 503 |
| 439 | 292 | 3.8 | 670 |
| 440 | 1012 | 4.4 | 1288 |
| 441 | 1059 | 3.8 | 1324 |
| 442 | 404 | 4.0 | 845 |
| 443 | 791 | 4.4 | 1161 |
| 444 | 343 | 2.8 | 795 |
| 445 | 913 | 4.0 | 1240 |
| 446 | 619 | 4.3 | 1052 |
| 447 | 1077 | 3.2 | 1329 |
| 448 | 736 | 4.2 | 1130 |
| 449 | 142 | 2.3 | 497 |
| 450 | 657 | 4.2 | 1078 |
| 451 | 373 | 4.4 | 825 |
| 452 | 730 | 4.2 | 1125 |
| 453 | 659 | 4.0 | 1081 |
| 454 | 1004 | 4.4 | 1282 |
| 455 | 1030 | 4.2 | 1296 |
| 456 | 314 | 2.8 | 723 |
| 457 | 830 | 4.3 | 1178 |
| 458 | 296 | 2.8 | 677 |
| 459 | 273 | 3.9 | 642 |
| 460 | 1153 | 3.8 | 1458 |
| 461 | 788 | 4.0 | 1159 |
| 462 | 593 | 4.9 | 1031 |
| 463 | 274 | 2.3 | 643 |
| 464 | 646 | 4.3 | 1074 |
| 465 | 536 | 3.3 | 985 |
| 466 | 1043 | 3.8 | 1300 |
| 467 | 1116 | 3.8 | 1379 |
| 468 | 1007 | 4.0 | 1286 |
| 469 | 991 | 4.0 | 1276 |
| 470 | 173 | 2.7 | 547 |
| 471 | 330 | 4.7 | 781 |
| 472 | 472 | 4.0 | 947 |
| 473 | 429 | 3.6 | 883 |
| 474 | 44 | 8.1 | 293 |
| 475 | 1102 | 4.2 | 1364 |
| 476 | 719 | 3.0 | 1122 |
| 477 | 326 | 4.0 | 773 |
| 478 | 836 | 4.2 | 1183 |
| 479 | 1037 | 4.5 | 1298 |
| 480 | 420 | 4.0 | 862 |
| 481 | 139 | 3.4 | 493 |
| 482 | 565 | 2.8 | 1007 |
| 483 | 746 | 4.0 | 1131 |
| 484 | 451 | 4.8 | 925 |
| 485 | 879 | 4.0 | 1217 |
| 486 | 840 | 4.4 | 1187 |
| 487 | 174 | 4.2 | 548 |
| 488 | 481 | 4.4 | 955 |
| 489 | 452 | 4.4 | 925 |
| 490 | 553 | 5.1 | 1001 |
| 491 | 648 | 2.8 | 1075 |
| 492 | 1110 | 3.7 | 1372 |
| 493 | 300 | 4.4 | 679 |
| 494 | 699 | 4.3 | 1113 |
| 495 | 93 | 3.0 | 409 |
| 496 | 1075 | 3.2 | 1329 |
| 497 | 200 | 7.2 | 564 |
| 498 | 971 | 4.0 | 1268 |
| 499 | 228 | 6.6 | 589 |
| 500 | 1089 | 4.3 | 1348 |
| 501 | 637 | 3.4 | 1070 |
| 502 | 615 | 4.3 | 1051 |
| 503 | 250 | 3.8 | 606 |
| 504 | 328 | 2.8 | 775 |
| 505 | 740 | 4.0 | 1130 |
| 506 | 1006 | 3.9 | 1284 |
| 507 | 966 | 4.3 | 1263 |
| 508 | 15 | 2.9 | 240 |
| 509 | 873 | 4.3 | 1216 |
| 510 | 572 | 2.2 | 1009 |
| 511 | 1055 | 3.8 | 1318 |
| 512 | 670 | 4.5 | 1093 |
| 513 | 220 | 2.7 | 579 |
| 514 | 1114 | 3.8 | 1379 |
| 515 | 760 | 4.4 | 1137 |
| 516 | 211 | 4.0 | 575 |
| 517 | 814 | 4.4 | 1169 |
| 518 | 757 | 4.2 | 1137 |
| 519 | 820 | 4.7 | 1171 |
| 520 | 946 | 2.8 | 1254 |
| 521 | 506 | 3.6 | 971 |
| 522 | 280 | 3.0 | 649 |
| 523 | 281 | 2.8 | 649 |
| 524 | 557 | 3.0 | 1003 |
| 525 | 578 | 4.3 | 1023 |
| 526 | 258 | 4.0 | 619 |
| 527 | 1028 | 4.5 | 1296 |
| 528 | 550 | 4.4 | 997 |
| 529 | 798 | 4.2 | 1163 |
| 530 | 393 | 4.1 | 841 |
| 531 | 1009 | 4.0 | 1286 |
| 532 | 697 | 2.8 | 1109 |
| 533 | 75 | 2.2 | 341 |
| 534 | 277 | 2.2 | 645 |
| 535 | 708 | 3.4 | 1117 |
| 536 | 968 | 4.5 | 1265 |
| 537 | 1060 | 4.2 | 1324 |
| 538 | 219 | 3.0 | 577 |
| 539 | 894 | 4.4 | 1226 |
| 540 | 201 | 7.0 | 564 |
| 541 | 11 | 2.7 | 240 |
| 542 | 1039 | 4.0 | 1300 |
| 543 | 824 | 4.0 | 1174 |
| 544 | 1083 | 3.8 | 1342 |
| 545 | 843 | 4.0 | 1192 |
| 546 | 864 | 4.0 | 1209 |
| 547 | 7 | 3.0 | 235 |
| 548 | 787 | 3.0 | 1157 |
| 549 | 639 | 3.4 | 1071 |
| 550 | 755 | 4.4 | 1137 |
| 551 | 958 | 3.8 | 1260 |
| 552 | 875 | 4.4 | 1216 |
| 553 | 1080 | 3.8 | 1338 |
| 554 | 512 | 3.8 | 973 |
| 555 | 1118 | 4.0 | 1388 |
| 556 | 461 | 2.8 | 931 |
| 557 | 267 | 3.8 | 629 |
| 558 | 483 | 4.3 | 955 |
| 559 | 108 | 3.0 | 455 |
| 560 | 45 | 6.6 | 297 |
| 561 | 949 | 4.0 | 1256 |
| 562 | 196 | 2.6 | 563 |
| 563 | 903 | 4.5 | 1232 |
| 564 | 1003 | 3.8 | 1282 |
| 565 | 777 | 4.2 | 1149 |
| 566 | 85 | 3.8 | 385 |
| 567 | 942 | 4.2 | 1252 |
| 568 | 906 | 4.5 | 1236 |
| 569 | 289 | 4.4 | 653 |
| 570 | 623 | 4.3 | 1059 |
| 571 | 878 | 4.0 | 1216 |
| 572 | 143 | 4.7 | 501 |
| 573 | 1005 | 3.8 | 1282 |
| 574 | 391 | 3.2 | 841 |
| 575 | 311 | 4.4 | 723 |
| 576 | 752 | 4.2 | 1135 |
| 577 | 339 | 2.8 | 791 |
| 578 | 704 | 3.4 | 1117 |
| 579 | 355 | 3.2 | 815 |
| 580 | 790 | 4.0 | 1160 |
| 581 | 850 | 4.3 | 1194 |
| 582 | 833 | 4.3 | 1180 |
| 583 | 286 | 2.5 | 653 |
| 584 | 724 | 4.3 | 1123 |
| 585 | 747 | 3.3 | 1133 |
| 586 | 62 | 5.7 | 325 |
| 587 | 416 | 3.8 | 861 |
| 588 | 616 | 2.9 | 1051 |
| 589 | 1051 | 3.6 | 1316 |
| 590 | 542 | 4.3 | 991 |
| 591 | 1046 | 3.8 | 1303 |
| 592 | 12 | 2.1 | 240 |
| 593 | 655 | 2.8 | 1077 |
| 594 | 130 | 2.2 | 489 |
| 595 | 607 | 3.8 | 1047 |
| 596 | 576 | 3.0 | 1017 |
| 597 | 162 | 2.8 | 531 |
| 598 | 371 | 3.4 | 823 |
| 599 | 195 | 3.4 | 561 |
| 600 | 370 | 3.9 | 823 |
| 601 | 940 | 4.4 | 1252 |
| 602 | 466 | 4.0 | 944 |
| 603 | 433 | 3.4 | 885 |
| 604 | 80 | 2.3 | 367 |
| 605 | 76 | 3.0 | 341 |
| 606 | 872 | 3.8 | 1213 |

(2) LC/MS Data Analysis for Metabolites of Ginseng Hairy Root

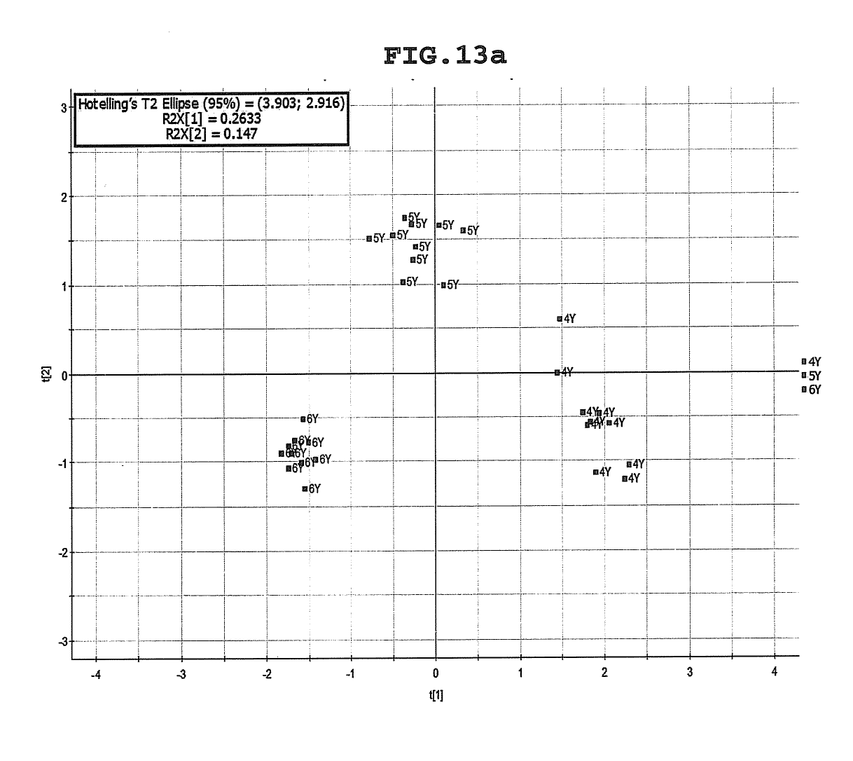

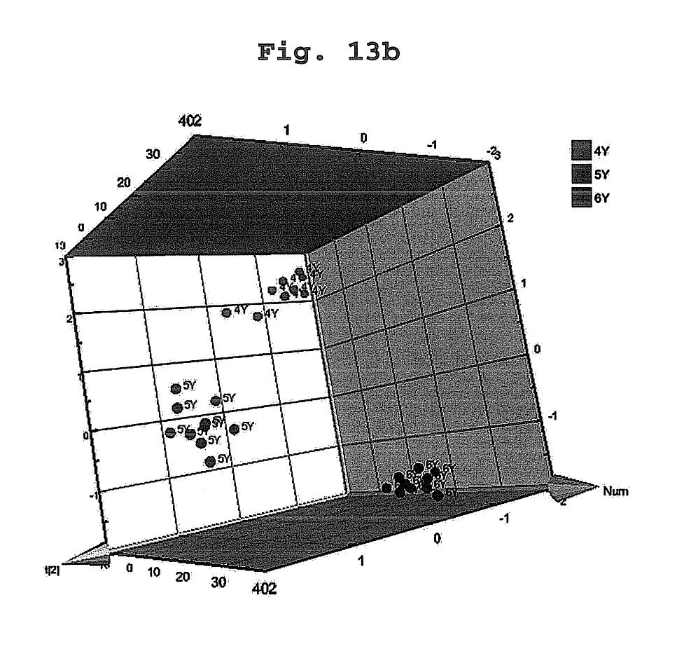

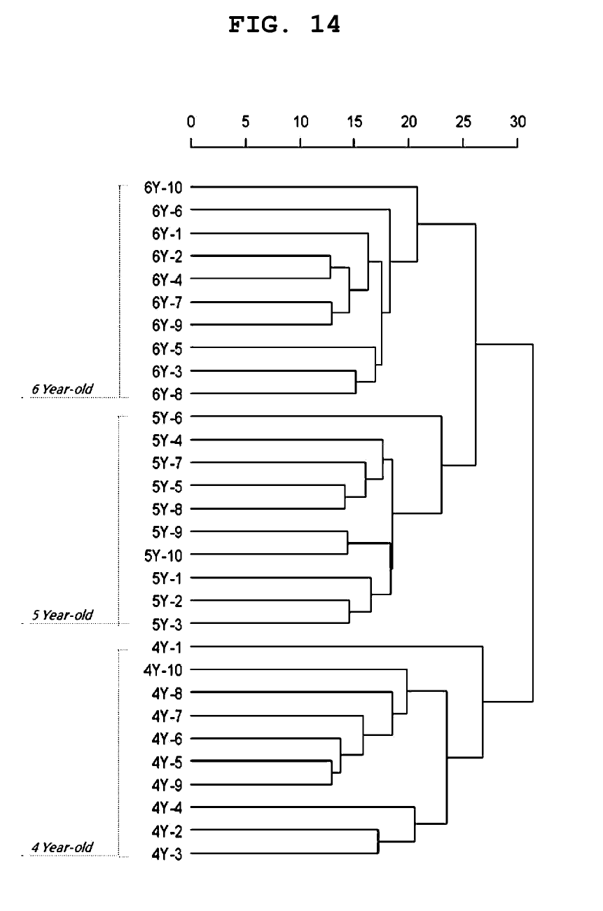

In contrast to the taproot data, the LC/MS data of the total metabolites of hairy roots allowed PCA and HCA to clearly separate 4- to 6-year-old ginseng roots from one another (FIGS. 13A, 13B, and 14).

The analysis data of metabolites from each of hairy roots at the age of 4 to 6 years was cross-validated for the classification accuracy of each age of ginseng roots using the three feature selection methods RF, PAM, and PLS-DA and was found to exactly classify the ages of ginseng roots (Tables 10 to 13). Table 10 summarizes the classification accuracy of hairy roots at the age of 4 to 6 years determined by RF, PAM, and PLS-DA. Tables 11 to 13 are confusion tables showing the prediction accuracy for ages of the hairy roots at the age of 4 to 6 years as analyzed by RF, PAM, and PLS-DA, respectively.

TABLE 10

| Classification | No. of selected | CV Accuracy (n = 30) | | | |
|---|---|---|---|---|---|
| method | metabolites | 4 Years | 5 Years | 6 Years | Mean |
| RF | 8 | 1.000 | 1.000 | 1.000 | 1.000 |
| PAM | 11 | 1.000 | 1.000 | 1.000 | 1.000 |
| PLA-DA | 16 | 1.000 | 1.000 | 1.000 | 1.000 |

TABLE 11

| | | Predicted Class | | | Classification |
|---|---|---|---|---|---|
| | | 4 Y | 5 Y | 6 Y | Accuracy |
| True | 4 Y | 500 | 0 | 0 | 1.000 |
| Class | 5 Y | 0 | 500 | 0 | 1.000 |
| | 6 Y | 0 | 0 | 500 | 1.000 |

TABLE 12

| | | Predicted Class | | | Classification |
|---|---|---|---|---|---|
| | | 4 Y | 5 Y | 6 Y | Accuracy |
| True | 4 Y | 500 | 0 | 0 | 1.000 |
| Class | 5 Y | 0 | 500 | 0 | 1.000 |
| | 6 Y | 0 | 0 | 500 | 1.000 |

TABLE 13

| | | Predicted Class | | | Classification |
|---|---|---|---|---|---|
| | | 4 Y | 5 Y | 6 Y | Accuracy |
| True | 4 Y | 500 | 0 | 0 | 1.000 |
| Class | 5 Y | 0 | 500 | 0 | 1.000 |
| | 6 Y | 0 | 0 | 500 | 1.000 |

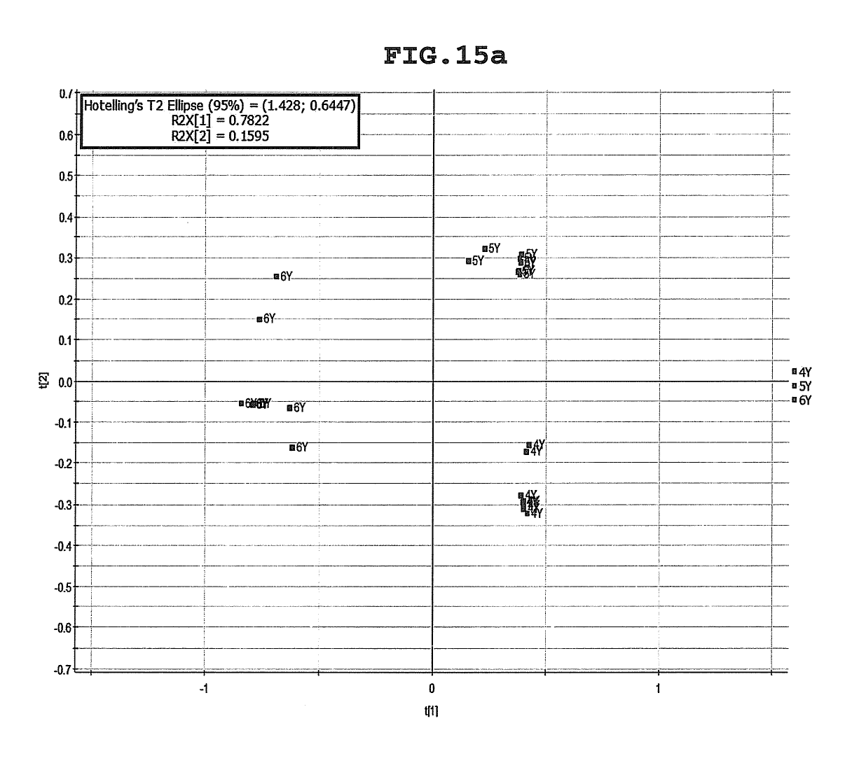

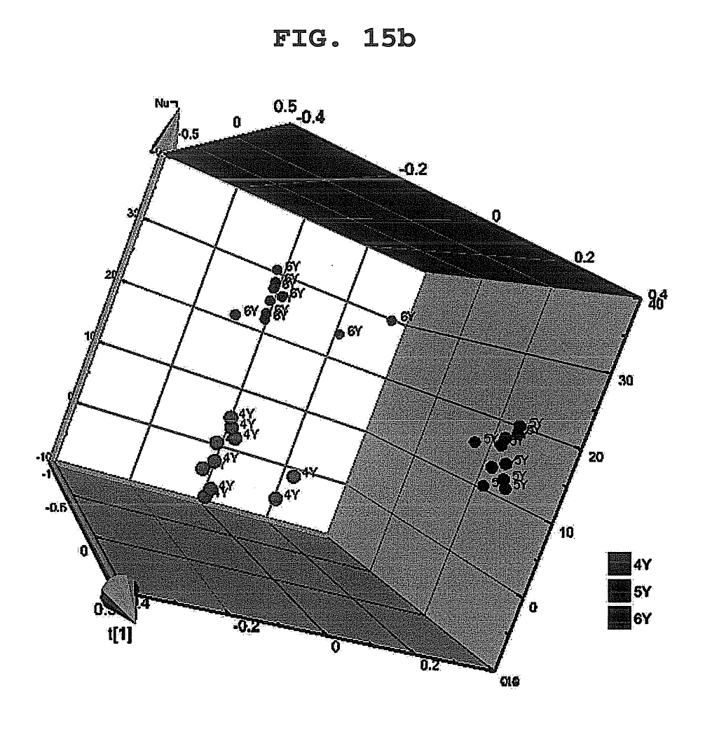

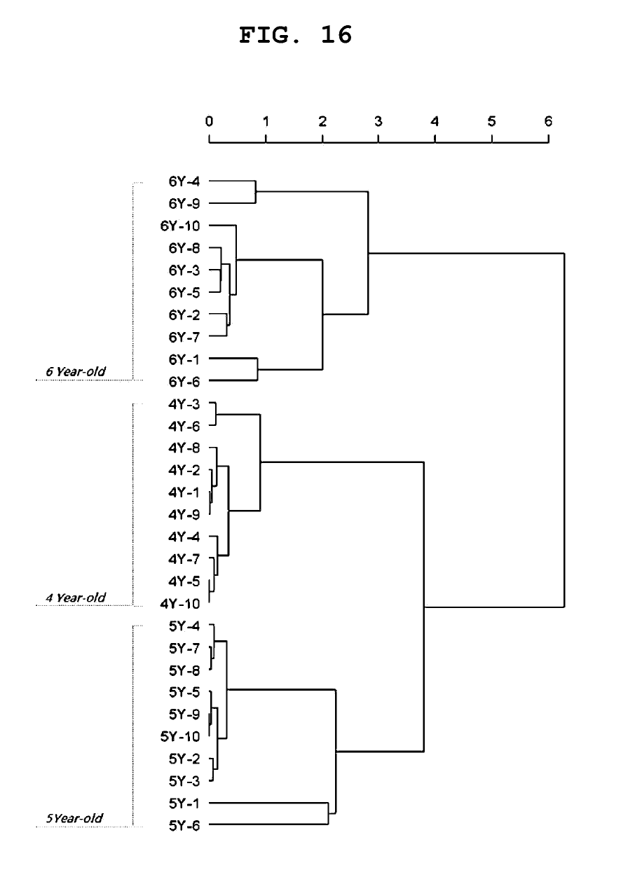

In addition, when evaluated with data of the 9 metabolites of ginseng hairy roots at the age of 4 to 6 years selected from the total metabolites by at least two of RF, PAM, and PLS-DA (Table 14), PCA and HCA were found to determine the ages of ginseng roots at the age of 4 to 6 with significance (FIGS. 15A, 15B, and 16).

TABLE 14

| | | Retention | Ion |
|---|---|---|---|
| NO | Meta # | Time (min.) | (m/z) |
| 1 | 152 | 4.1 | 1105 |
| 2 | 244 | 3.2 | 1436 |
| 3 | 121 | 4.4 | 971 |
| 4 | 35 | 2.2 | 499 |
| 5 | 108 | 3.6 | 883 |
| 6 | 122 | 4.4 | 971 |
| 7 | 82 | 4.2 | 841 |
| 8 | 164 | 4.3 | 1143 |
| 9 | 109 | 2.9 | 861 |

### Example 5

### GC/MS Data Analysis for Determination of Ginseng Age

1) GC/MS Analysis Results

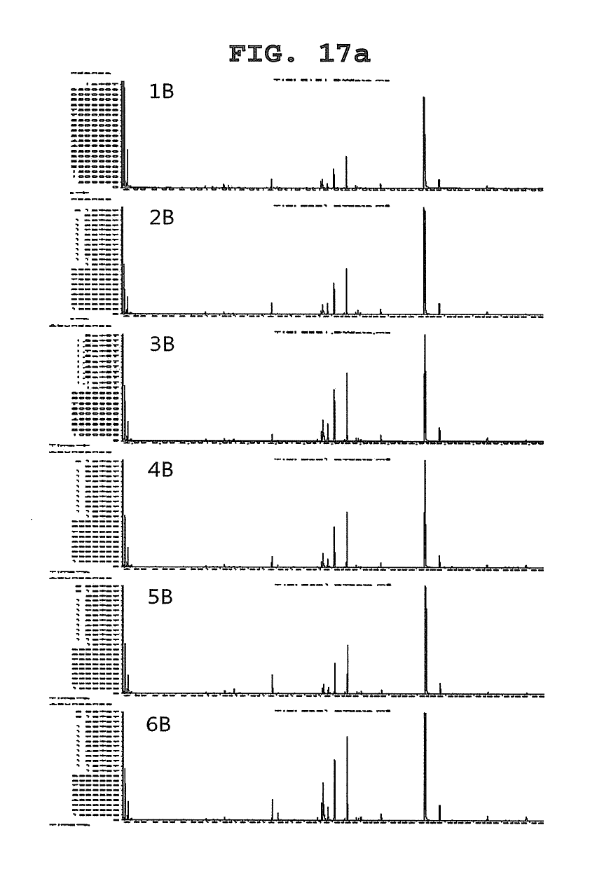

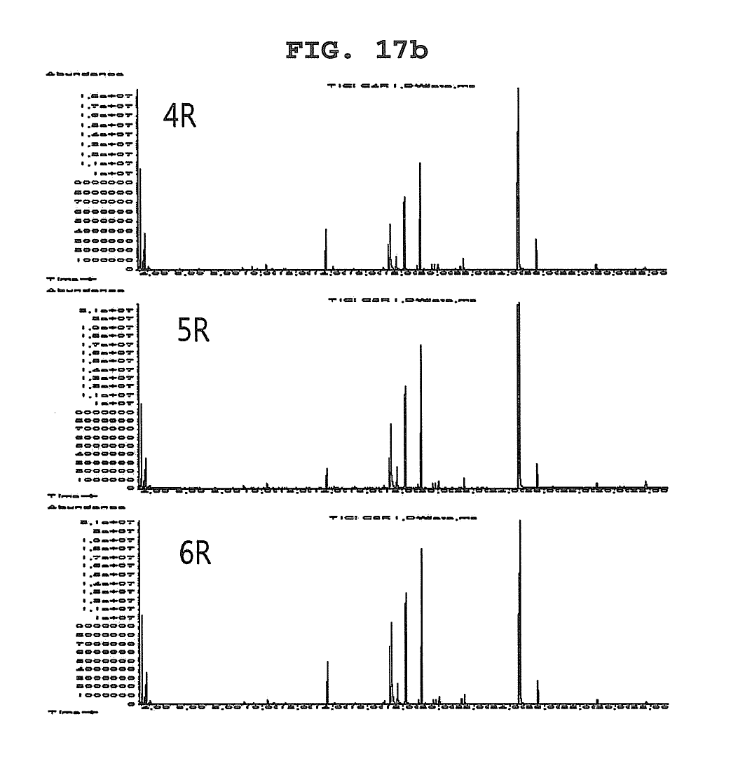

It was difficult to discriminate different ages with the GC/MS data obtained for each of the taproots and hairy roots (FIGS. 17A and 17B).

2) Statics of GC/MS Data (Chemometric Analysis)

With GC/MS data for each of the taproots and the hairy roots, PCA and HCA were preformed. PCA is an unsupervised clustering method, most widely used among multivariate statistical analysis methods, by which a difference between experimental groups can be identified, while HCA is a method by which subjects are classified into clusters and a hierarchy of clusters is built to establish relationships therebetween.

(1) GC/MS Data Analysis for Metabolites of Ginseng Taproot

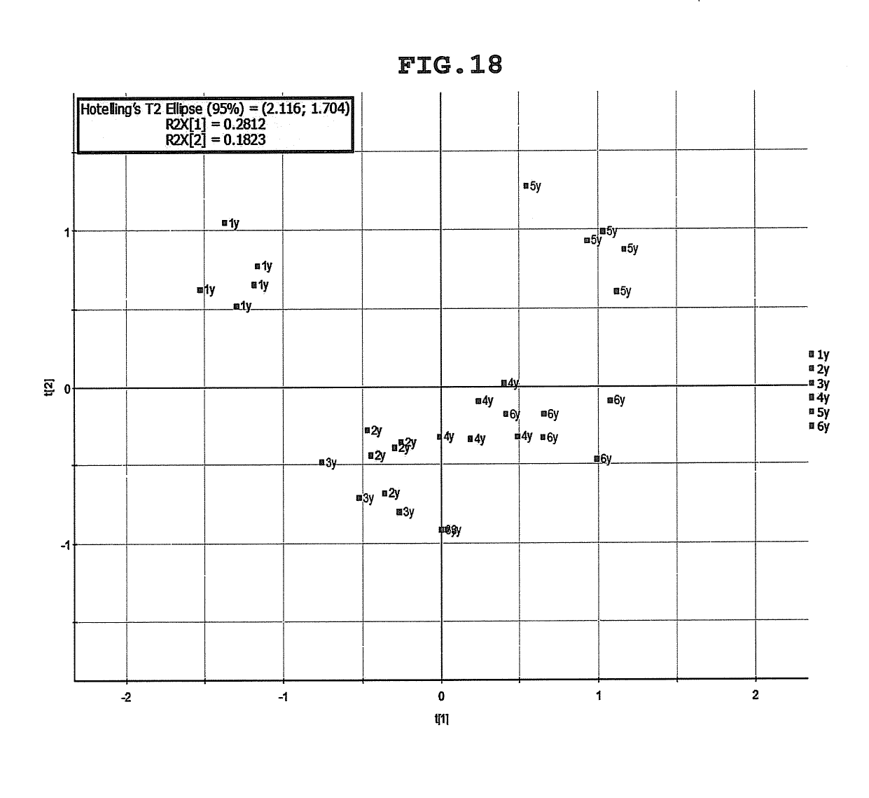

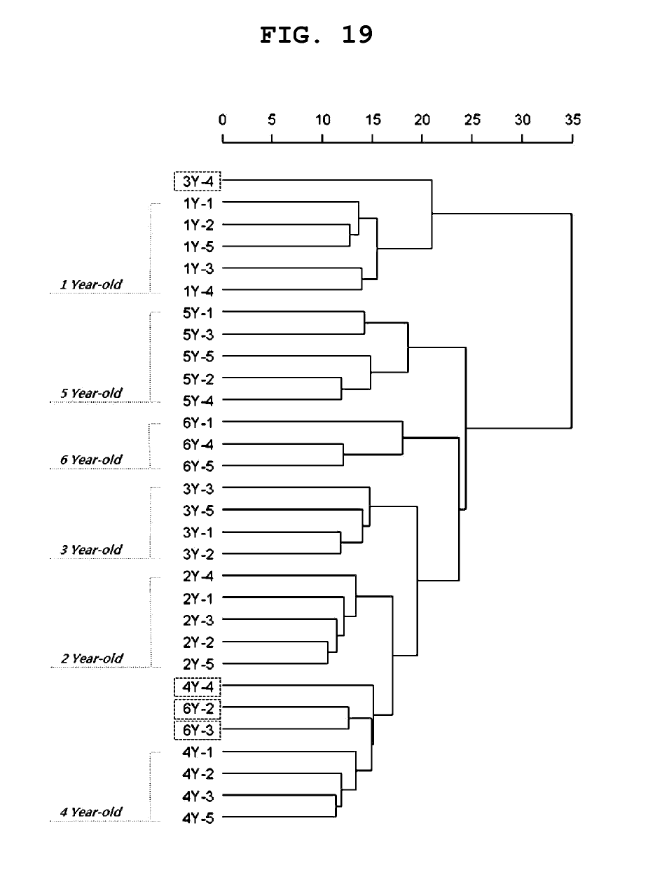

With data about metabolites of each taproot at the age of 1 to 6 years, PCA and HCA were preformed. As a result, merely the raw GC/MS data of ginseng taproots was sufficient to discriminate ginseng roots at the age of 1 and 5 years (FIGS. 18 and 19).

The analysis data for metabolites of each taproot at the age of 1 to 6 years was cross-validated for the classification accuracy of each age of ginseng roots using the three feature selection methods RF, PAM, and PLS-DA, and was found to classify the ages of ginseng roots with approximately 80% accuracy (Tables 15 to 18). Table 15 summarizes the classification accuracy of taproots at the age of 1 to 6 years determined by RF, PAM, and PLS-DA. Tables 16 to 18 are confusion tables showing the prediction accuracy for ages of the taproots at the age of 1 to 6 years as analyzed by RF, PAM, and PLS-DA, respectively.

TABLE 15

| Classi- | No. of | CV Accuracy (n = 30) | | | | | | |
|---|---|---|---|---|---|---|---|---|
| fication | selected | 1 | 2 | 3 | 4 | 5 | 6 | |
| method | metabolites | Year | Years | Years | Years | Years | Years | Mean |
| RF | 50 | 1.000 | 0.972 | 0.786 | 0.690 | 1.000 | 0.736 | 0.864 |
| PAM | 185 | 1.000 | 0.970 | 0.790 | 0.992 | 1.000 | 0.796 | 0.925 |
| PLA- | 37 | 1.000 | 1.000 | 0.920 | 0.996 | 1.000 | 1.000 | 0.986 |
| DA | | | | | | | | |

TABLE 16

| | | Predicted Class | | | | | | Classification |
|---|---|---|---|---|---|---|---|---|
| | | 1 Y | 2 Y | 3 Y | 4 Y | 5 Y | 6 Y | Accuracy |
| True | 1 Y | 500 | 0 | 0 | 0 | 0 | 0 | 1.000 |
| Class | 2 Y | 0 | 486 | 8 | 6 | 0 | 0 | 0.972 |
| | 3 Y | 0 | 107 | 393 | 0 | 0 | 0 | 0.786 |
| | 4 Y | 0 | 0 | 0 | 345 | 0 | 155 | 0.690 |
| | 5 Y | 0 | 0 | 0 | 0 | 500 | 0 | 1.000 |
| | 6 Y | 0 | 0 | 1 | 121 | 10 | 368 | 0.736 |

TABLE 17

| | | Predicted Class | | | | | | Classification |
|---|---|---|---|---|---|---|---|---|
| | | 1 Y | 2 Y | 3 Y | 4 Y | 5 Y | 6 Y | Accuracy |
| True | 1 Y | 500 | 0 | 0 | 0 | 0 | 0 | 1.000 |
| Class | 2 Y | 0 | 485 | 15 | 0 | 0 | 0 | 0.970 |
| | 3 Y | 0 | 105 | 395 | 0 | 0 | 0 | 0.790 |
| | 4 Y | 0 | 0 | 0 | 496 | 0 | 4 | 0.992 |
| | 5 Y | 0 | 0 | 0 | 0 | 500 | 0 | 1.000 |
| | 6 Y | 0 | 0 | 0 | 101 | 1 | 398 | 0.796 |

TABLE 18

| | | Predicted Class | | | | | | Classification |
|---|---|---|---|---|---|---|---|---|
| | | 1 Y | 2 Y | 3 Y | 4 Y | 5 Y | 6 Y | Accuracy |
| True | 1 Y | 500 | 0 | 0 | 0 | 0 | 0 | 1.000 |
| Class | 2 Y | 0 | 500 | 0 | 0 | 0 | 0 | 1.000 |
| | 3 Y | 0 | 40 | 460 | 0 | 0 | 0 | 0.920 |
| | 4 Y | 0 | 0 | 0 | 498 | 0 | 2 | 0.996 |
| | 5 Y | 0 | 0 | 0 | 0 | 500 | 0 | 1.000 |
| | 6 Y | 0 | 0 | 0 | 0 | 0 | 500 | 1.000 |

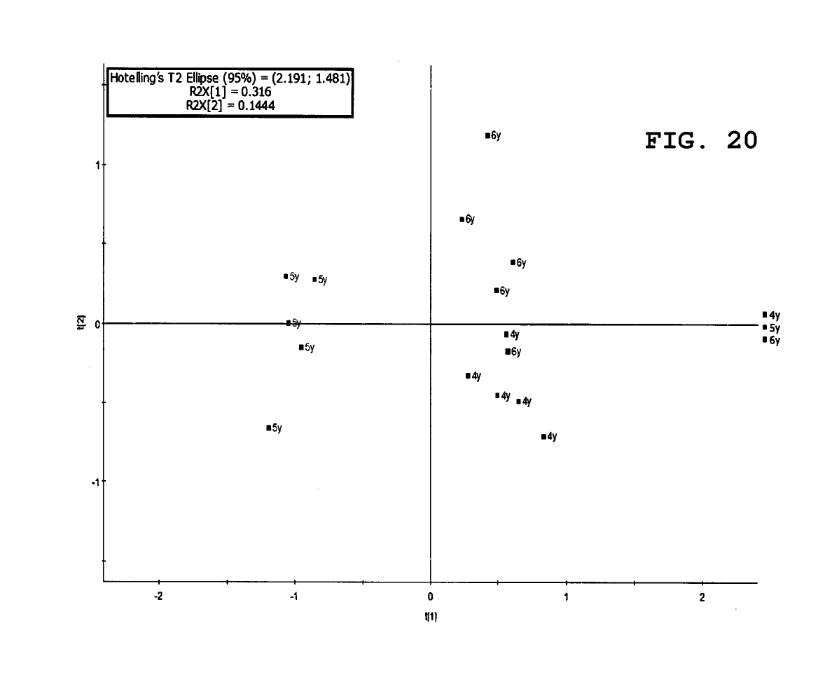

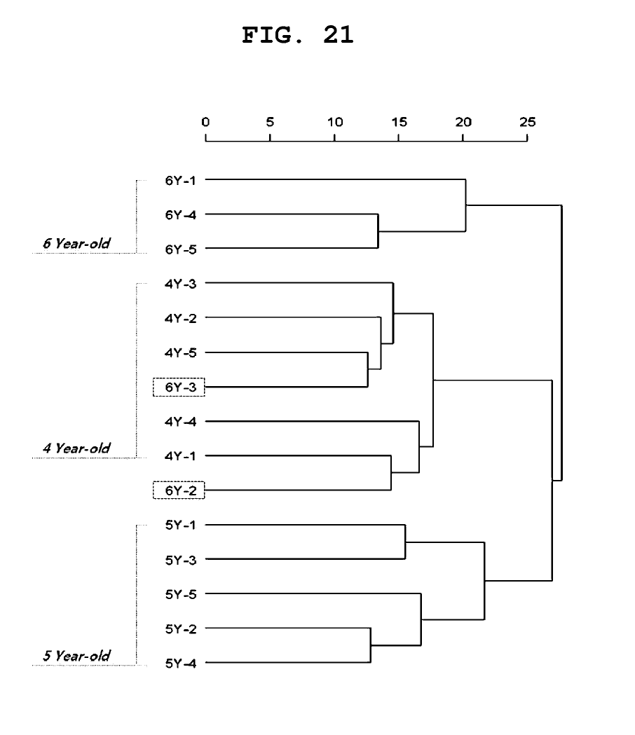

Only with the data of 4- to 6-year-old ginseng roots, which are of main interest to the present invention, was the above statistical analysis performed. As a result, it was rather difficult to exactly discriminate the ginseng roots at the age of 4 to 6 years with the data of total metabolites (FIGS. 20 and 21).

However, the data of 4- to 6-year-old ginseng taproots were found to allow for the determination of ages of 4- to 6-year-old ginseng roots as analyzed by the three feature selection methods RF, PAM, and PLS-DA, with perfect discrimination by PLS-DA (Tables 19 to 22). Table 19 summarizes the classification accuracy of taproots at the age of 4 to 6 years determined by RF, PAM, and PLS-DA. Tables 20 to 22 are confusion tables showing the prediction accuracy for ages of the 4- to 6-year-old taproots as analyzed by RF, PAM, and PLS-DA, respectively.

TABLE 19

| Classification | No. of selected | CV Accuracy (n = 15) | | | |
|---|---|---|---|---|---|
| method | metabolites | 4 Years | 5 Years | 6 Years | Mean |
| RF | 25 | 1.000 | 1.000 | 0.800 | 0.933 |
| PAM | 150 | 0.976 | 1.000 | 0.796 | 0.924 |
| PLA-DA | 31 | 1.000 | 1.000 | 1.000 | 1.000 |

TABLE 20

| | | Predicted Class | | | Classification |
|---|---|---|---|---|---|
| | | 4 Y | 5 Y | 6 Y | Accuracy |
| True | 4 Y | 250 | 0 | 0 | 1.000 |
| Class | 5 Y | 0 | 250 | 0 | 1.000 |
| | 6 Y | 50 | 0 | 200 | 0.800 |

TABLE 21

| | | Predicted Class | | | Classification |
|---|---|---|---|---|---|
| | | 4 Y | 5 Y | 6 Y | Accuracy |
| True | 4 Y | 244 | 0 | 6 | 0.976 |
| Class | 5 Y | 0 | 250 | 0 | 1.000 |
| | 6 Y | 51 | 0 | 199 | 0.796 |

TABLE 22

| | | Predicted Class | | | Classification |
|---|---|---|---|---|---|
| | | 4 Y | 5 Y | 6 Y | Accuracy |
| True | 4 Y | 250 | 0 | 0 | 1.000 |
| Class | 5 Y | 0 | 250 | 0 | 1.000 |
| | 6 Y | 0 | 0 | 250 | 1.000 |

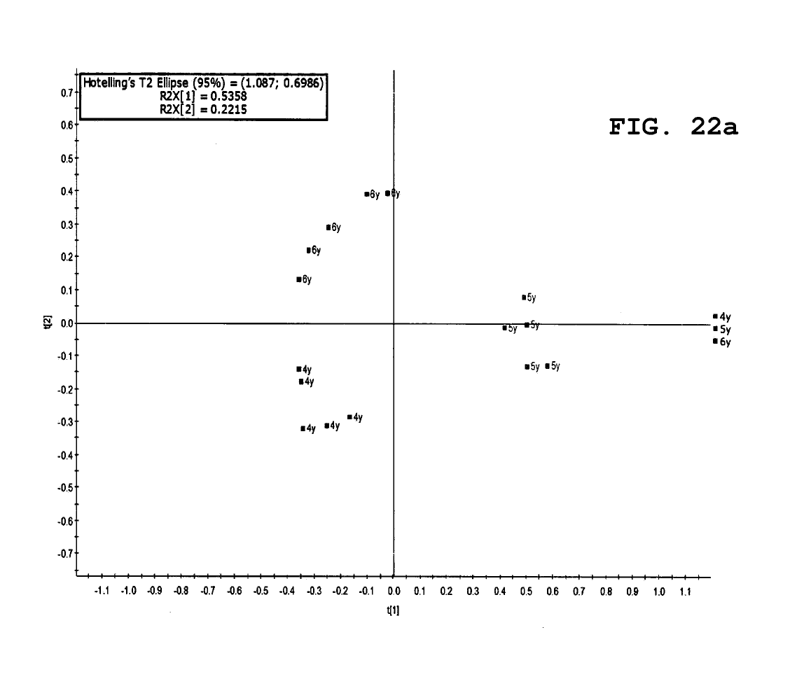

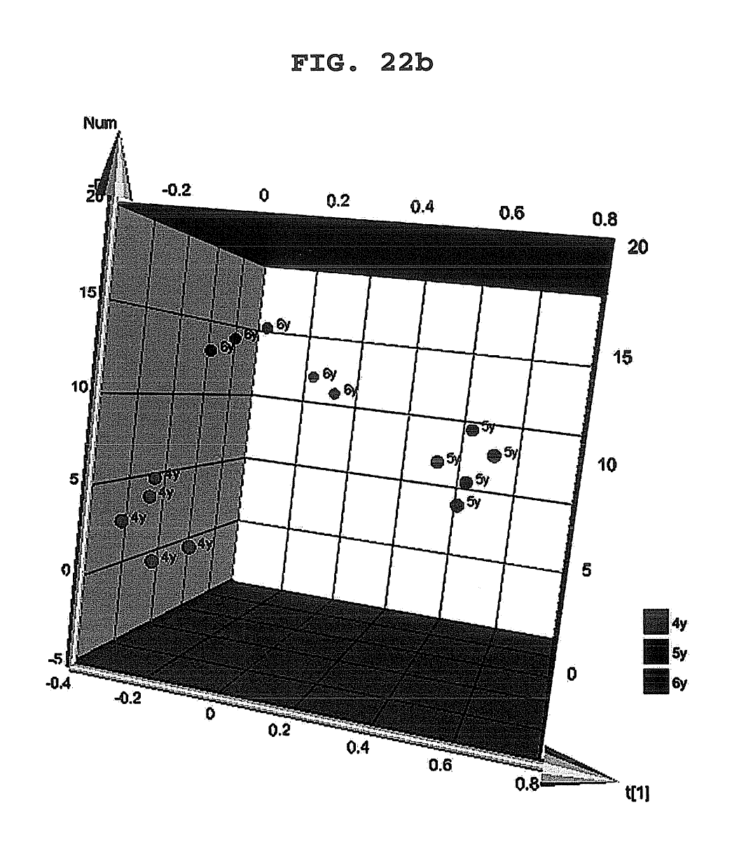

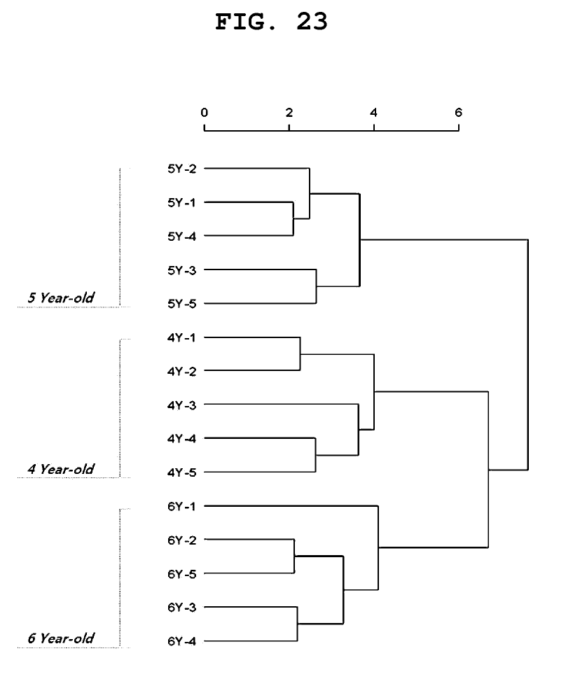

In addition, when evaluated with data of the 13 metabolites of ginseng taproots at the age of 4 to 6 years commonly selected by all RF, PAM, and PLS-DA (Table 23), PCA and HCA was found to determine the exact ages of ginseng roots at the age of 4 to 6 (FIGS. 22A, 22B, and 23).

TABLE 23

| | | Retention | Ion | |
|---|---|---|---|---|
| NO | Meta # | Time (min.) | (m/z) | NIST Library |
| 1 | 117 | 4.0 | 105 | — |
| 2 | 157 | 20.1 | 73 | — |
| 3 | 139 | 21.3 | 73 | trimethylsilyl 1- |
| | | | | trimethylsilyl-5- |
| | | | | trimethylsiloxy-3-(2- |
| | | | | trimethylsilylamino)in- |
| | | | | dolepropionate |
| 4 | 140 | 29.8 | 217 | — |
| 5 | 168 | 27.9 | 217 | — |
| 6 | 199 | 15.5 | 217 | — |
| 7 | 148 | 9.5 | 73 | — |
| 8 | 185 | 12.5 | 287 | — |
| 9 | 172 | 13.8 | 57 | — |
| 10 | 65 | 3.4 | 70 | 2-Thiazolidinone, |
| | | | | 3-(1-methylethyl)- |
| | | | | 4-methyl- |
| 11 | 105 | 21.2 | 75 | 9,12-Octadecadienoic acid |
| | | | | (Z,Z)-, trimethylsilyl ester |
| 12 | 40 | 13.7 | 73 | — |
| 13 | 162 | 14.3 | 71 | — |

(2) GC/MS Data Analysis for Metabolites of Ginseng Hairy Root

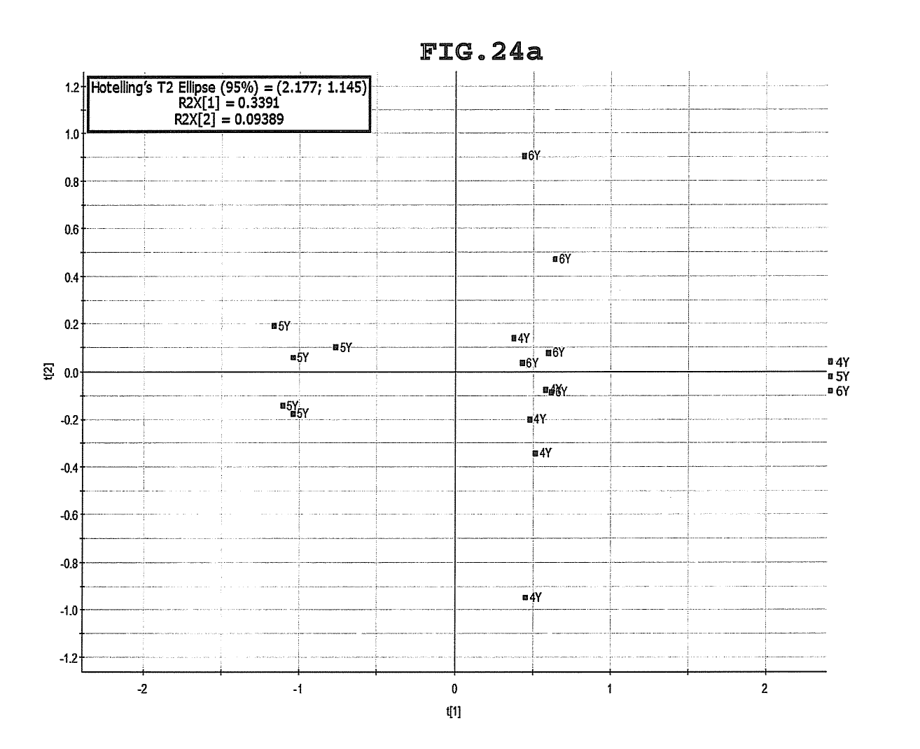

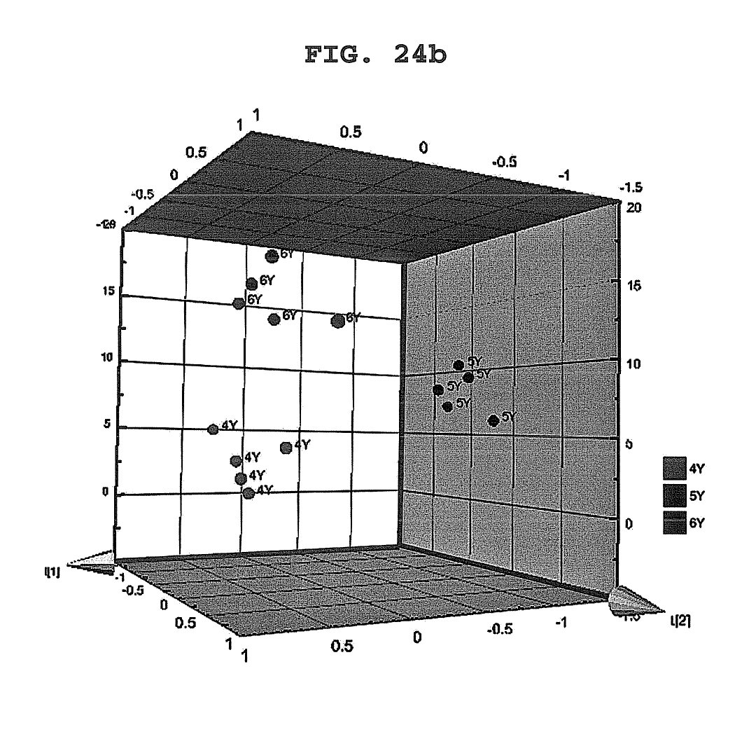

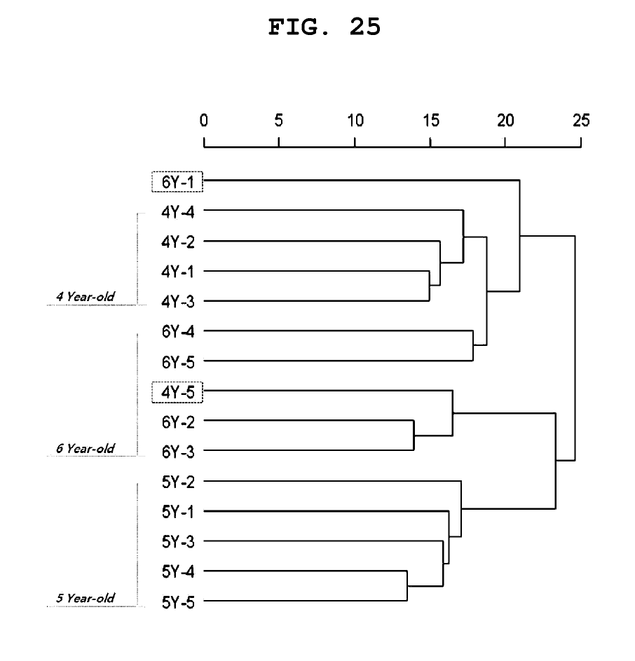

In contrast to the taproot data, it was rather difficult to perfectly discriminate ginseng roots at the age of 4 to 6 years by performing PCA and HCA with the GC/MS data of the total metabolites of hairy roots (FIGS. 24A, 24B, and 25).

The analysis data of metabolites from each of hairy roots at the age of 4 to 6 years was cross-validated for the classification accuracy of each age of ginseng roots using the three feature selection methods RF, PAM, and PLS-DA and was found to exactly classify the ages of ginseng roots (Tables 24 to 27). Table 24 summarizes the classification accuracy of hairy roots at the age of 4 to 6 years determined by RF, PAM, and PLS-DA. Tables 25 to 27 are confusion tables showing the prediction accuracy for ages of the hairy roots at the age of 4 to 6 years as analyzed by RF, PAM, and PLS-DA, respectively.

TABLE 24

| Classification | No. of selected | CV Accuracy (n = 15) | | | |
|---|---|---|---|---|---|
| method | metabolites | 4 Years | 5 Years | 6 Years | Mean |
| RF | 7 | 0.988 | 1.000 | 0.836 | 0.941 |
| PAM | 56 | 0.832 | 1.000 | 0.828 | 0.887 |
| PLA-DA | 19 | 1.000 | 1.000 | 1.000 | 1.000 |

TABLE 25

| | | Predicted Class | | | Classification |
|---|---|---|---|---|---|
| | | 4 Y | 5 Y | 6 Y | Accuracy |
| True | 4 Y | 247 | 0 | 3 | 0.988 |
| Class | 5 Y | 0 | 250 | 0 | 1.000 |
| | 6 Y | 41 | 0 | 209 | 0.836 |

TABLE 26

| | | Predicted Class | | | Classification |
|---|---|---|---|---|---|
| | | 4 Y | 5 Y | 6 Y | Accuracy |
| True | 4 Y | 208 | 0 | 42 | 0.832 |
| Class | 5 Y | 0 | 250 | 0 | 1.000 |
| | 6 Y | 43 | 0 | 207 | 0.828 |

TABLE 27

| | | Predicted Class | | | Classification |
|---|---|---|---|---|---|
| | | 4 Y | 5 Y | 6 Y | Accuracy |
| True | 4 Y | 250 | 0 | 0 | 1.000 |
| Class | 5 Y | 0 | 250 | 0 | 1.000 |
| | 6 Y | 0 | 0 | 250 | 1.000 |

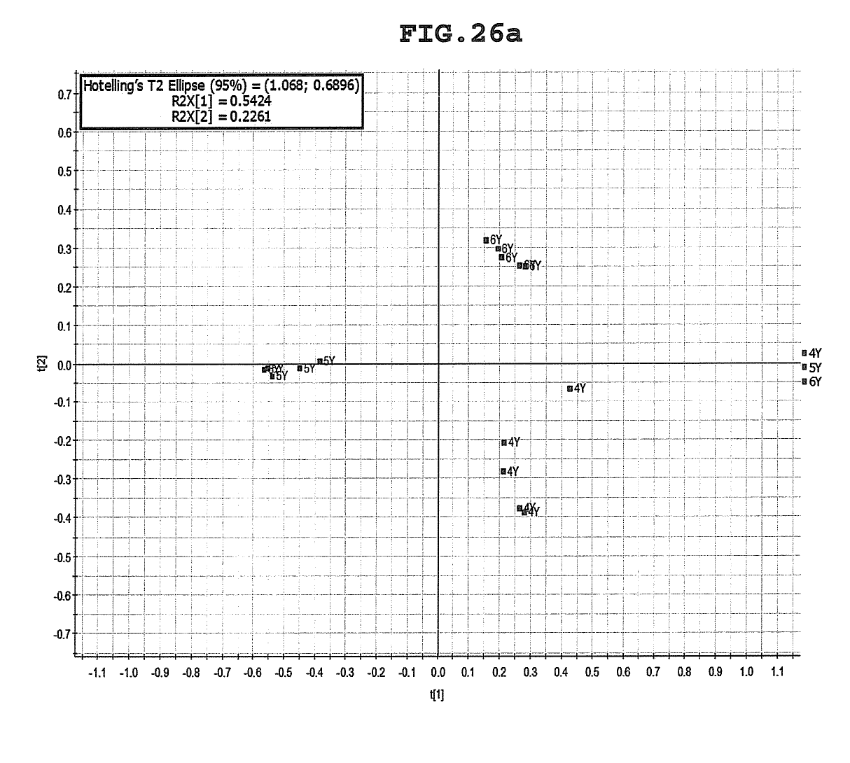

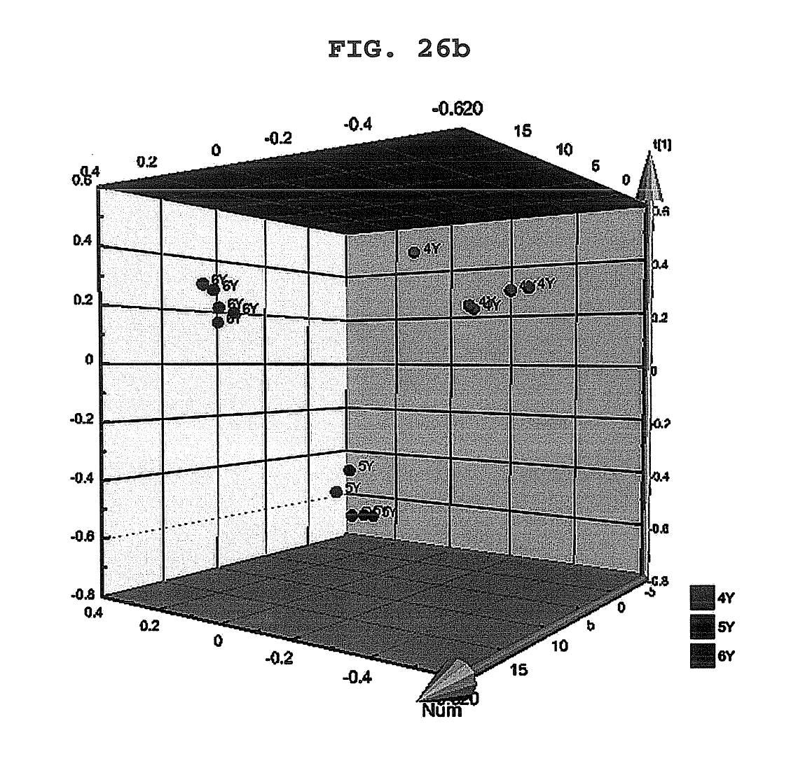

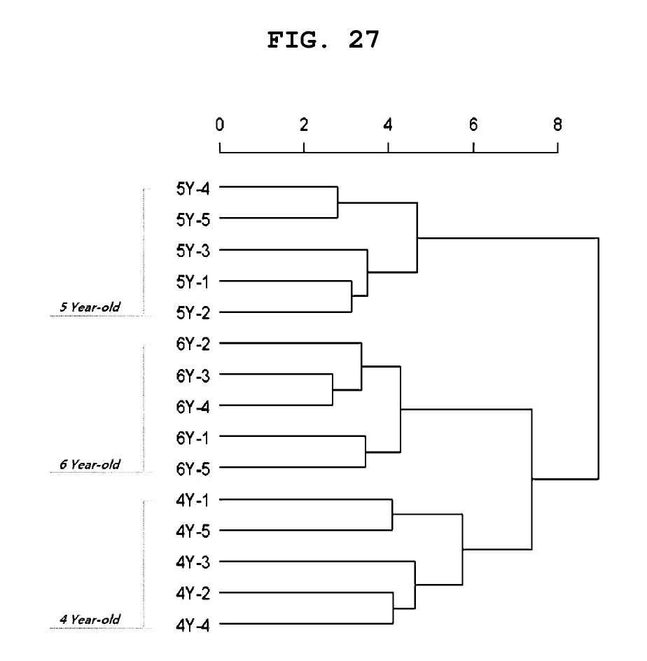

In addition, when evaluated with data of 21 metabolites commonly selected from the total metabolites of ginseng hairy roots at the age of 4 to 6 years by at least two of RF, PAM, and PLS-DA (Table 28), PCA and HCA was found to determine the ages of ginseng roots at the age of 4 to 6 with significance (FIGS. 26A, 26B, and 27).

TABLE 28

| | | Retention | Ion | |
|---|---|---|---|---|
| NO | Meta # | Time (min.) | (m/z) | NIST Library |
| 1 | 183 | 16.4 | 73 | — |
| 2 | 106 | 26.0 | 204 | — |
| 3 | 148 | 9.5 | 73 | — |
| 4 | 192 | 20.8 | 204 | Urea, N,N′- |
| | | | | bis(trimethylsilyl)- |
| 5 | 60 | 26.5 | 73 | — |
| 6 | 14 | 6.2 | 57 | — |
| 7 | 4 | 3.4 | 244 | — |
| 8 | 64 | 31.8 | 217 | αD-Glucopyranoside, |
| | | | | 1,3,4,6-tetrakis- |
| | | | | O-(trimethylsilyl)-βD- |
| | | | | fructofuranosyl 2,3,4,6- |
| | | | | tetrakis-O-(trimethylsilyl)- |
| 9 | 197 | 9.5 | 147 | — |
| 10 | 36 | 11.3 | 147 | Butanedioic acid, |
| | | | | bis(trimethylsilyl) |
| | | | | ester |
| 11 | 187 | 22.4 | 73 | dl-2-Benzylaminooctanol |
| 12 | 47 | 19.0 | 149 | — |
| 13 | 200 | 16.6 | 71 | — |
| 14 | 68 | 7.5 | 57 | 3-Ethyl-3-methylheptane |
| 15 | 9 | 3.7 | 171 | Silanamine, N,N′- |
| | | | | methanetetraylbis[1,1,1- |
| | | | | trimethyl- |
| 16 | 188 | 32.0 | 441 | — |
| 17 | 175 | 30.5 | 217 | — |
| 18 | 123 | 18.3 | 73 | — |
| 19 | 151 | 12.7 | 73 | — |
| 20 | 29 | 9.8 | 133 | — |
| 21 | 35 | 11.0 | 142 | L-Proline, 1-(trimethylsilyl)-, |
| | | | | trimethylsilyl ester |

Although the preferred embodiments of the present invention have been disclosed for illustrative purposes, those skilled in the art will appreciate that various modifications, additions, and substitutions are possible, without departing from the scope and spirit of the invention as disclosed in the accompanying claims.

1. A method for determining an age of ginseng roots using chromatography-mass spectroscopy, comprising:
extracting a metabolome from a ginseng sample;
subjecting the metabolome to liquid chromatography-mass spectroscopy (LC/MS) to afford an analysis result;
converting the LC/MS analysis result to statistically accessible data; and
performing a statistical analysis of the data to determine the age of ginseng sample.

2. The method of claim 1, wherein the statistical analysis is principal component analysis (PCA) or hierarchical cluster analysis (HCA).

3. The method of claim 1, wherein the ginseng sample is a taproot and is used to determine the ages of 1- to 3-year-old ginseng roots.

4. The method of claim 1, wherein the ginseng sample is a hairy root and is used to determine the ages of 4- to 6-year-old ginseng roots.

5. The method of claim 1, further comprising executing feature selection to select and analyze an influential and significant metabolite of different ages among the metabolome.

6. The method of claim 5, wherein the ginseng sample is a taproot and the feature selection is executed using PLS-DA (Partial Least Squares-Discriminant Analysis) to select and analyze a metabolite whereby the ages of 4- to 6-year-old ginseng roots can be determined.

7. The method of claim 5, wherein the ginseng sample is a taproot and the feature selection is executed using at least two selected from the group consisting of RF (Random Forest), PAM (Prediction Analysis for Microarray) and PLS-DA (Partial Least Squares-Discriminant Analysis) to select and analyze a common metabolite, whereby the ages of 4- to 6-year-old ginseng roots can be determined.

8. The method of claim 5, wherein the ginseng sample is a hairy root and the feature selection is executed using at least one selected from the group consisting of RF (Random Forest), PAM (Prediction Analysis for Microarray), and PLS-DA (Partial Least Squares-Discriminant Analysis) to select and analyze a metabolite, whereby the ages of 4- to 6-year-old ginseng roots can be determined.

9. The method of claim 5, wherein the ginseng sample is a hairy root and the feature selection is executed using at least two selected from the group consisting of RF (Random Forest), PAM (Prediction Analysis for Microarray), and PLS-DA (Partial Least Squares-Discriminant Analysis) to select and analyze a common metabolite, whereby the ages of 4- to 6-year-old ginseng roots can be determined.

10. An apparatus for determining ages of ginseng roots, comprising:
a memory for storing standard data of metabolites selected from a ginseng sample, said standard data comprising retention times and molecular weights of the metabolites and being pre-constructed by liquid chromatography-mass spectroscopy; and
an analysis means for comparing data measured for a ginseng sample of interest to the standard data, said data measured for the ginseng sample of interest being obtained by liquid chromatography-mass spectroscopy and comprising retention times and molecular weights of the same metabolites as said metabolites.

11. A method for determining an age of ginseng roots using chromatography-mass spectroscopy, comprising:
extracting a metabolome from a ginseng sample;
subjecting the metabolome to gas chromatography-mass spectroscopy (GC/MS) to afford an analysis result;
converting the GC/MS analysis result to statistically accessible data; and
performing a statistical analysis of the data to determine the age of ginseng sample.

12. The method of claim 11, wherein the statistical analysis is principal component analysis (PCA) or hierarchical cluster analysis (HCA).

13. The method of claim 11, wherein the ginseng sample is a taproot and is used to determine the ages of 1- or 5-year-old ginseng roots.

14. The method of claim 11, further comprising executing feature selection to select and analyze an influential and significant metabolite of different ages among the metabolome.

15. The method of claim 14, wherein the ginseng sample is a taproot and the feature selection is executed using PLS-DA (Partial Least Squares-Discriminant Analysis) to select and analyze a metabolite, whereby the ages of 4- to 6-year-old ginseng roots can be determined.

16. The method of claim 14, wherein the ginseng sample is a taproot and the feature selection is executed using RF (Random Forest), PAM (Prediction Analysis for Microarray), and PLS-DA (Partial Least Squares-Discriminant Analysis) to select and analyze a common metabolite, whereby the ages of 4- to 6-year-old ginseng roots can be determined.

17. The method of claim 14, wherein the ginseng sample is a hairy root and the feature selection is executed using PLS-DA (Partial Least Squares-Discriminant Analysis) to select and analyze a metabolite, whereby the ages of 4- to 6-year-old ginseng roots can be determined.

18. The method of claim 14, wherein the ginseng sample is a hairy root and the feature selection is executed using at least two selected from the group consisting of RF (Random Forest), PAM (Prediction Analysis for Microarray), and PLS-DA (Partial Least Squares-Discriminant Analysis) to select and analyze a common metabolite, whereby the ages of 4- to 6-year-old ginseng roots can be determined.

19. An apparatus for determining ages of ginseng roots, comprising:
a memory for storing standard data of metabolites selected from a ginseng sample, said standard data comprising retention times and molecular weights of the metabolites and being pre-constructed by gas chromatography-mass spectroscopy; and
an analysis means for comparing data measured for a ginseng sample of interest to the standard data, said data measured for the ginseng sample of interest being obtained by gas chromatography-mass spectroscopy and comprising retention times and molecular weights of the same metabolites as said metabolites.

20. The method of claim 2, wherein the ginseng sample is a taproot and is used to determine the ages of 1- to 3-year-old ginseng roots.

21. The method of claim 2, wherein the ginseng sample is a hairy root and is used to determine the ages of 4- to 6-year-old ginseng roots.

22. The method of claim 2, further comprising executing feature selection to select and analyze an influential and significant metabolite of different ages among the metabolome.

23. The method of claim 22, wherein the ginseng sample is a taproot and the feature selection is executed using PLS-DA (Partial Least Squares-Discriminant Analysis) to select and analyze a metabolite whereby the ages of 4- to 6-year-old ginseng roots can be determined.

24. The method of claim 22, wherein the ginseng sample is a taproot and the feature selection is executed using at least two selected from the group consisting of RF (Random Forest), PAM (Prediction Analysis for Microarray) and PLS-DA (Partial Least Squares-Discriminant Analysis) to select and analyze a common metabolite, whereby the ages of 4- to 6-year-old ginseng roots can be determined.

25. The method of claim 22, wherein the ginseng sample is a hairy root and the feature selection is executed using at least one selected from the group consisting of RF (Random Forest), PAM (Prediction Analysis for Microarray), and PLS-DA (Partial Least Squares-Discriminant Analysis) to select and analyze a metabolite, whereby the ages of 4- to 6-year-old ginseng roots can be determined.

26. The method of claim 22, wherein the ginseng sample is a hairy root and the feature selection is executed using at least two selected from the group consisting of RF (Random Forest), PAM (Prediction Analysis for Microarray), and PLS-DA (Partial Least Squares-Discriminant Analysis) to select and analyze a common metabolite, whereby the ages of 4- to 6-year-old ginseng roots can be determined.

27. The method of claim 12, wherein the ginseng sample is a taproot and is used to determine the ages of 1- or 5-year-old ginseng roots.

28. The method of claim 12, further comprising executing feature selection to select and analyze an influential and significant metabolite of different ages among the metabolome.

29. The method of claim 28, wherein the ginseng sample is a taproot and the feature selection is executed using PLS-DA (Partial Least Squares-Discriminant Analysis) to select and analyze a metabolite, whereby the ages of 4- to 6-year-old ginseng roots can be determined.

30. The method of claim 28, wherein the ginseng sample is a taproot and the feature selection is executed using RF (Random Forest), PAM (Prediction Analysis for Microarray), and PLS-DA (Partial Least Squares-Discriminant Analysis) to select and analyze a common metabolite, whereby the ages of 4- to 6-year-old ginseng roots can be determined.

31. The method of claim 28, wherein the ginseng sample is a hairy root and the feature selection is executed using PLS-DA (Partial Least Squares-Discriminant Analysis) to select and analyze a metabolite, whereby the ages of 4- to 6-year-old ginseng roots can be determined.

32. The method of claim 28, wherein the ginseng sample is a hairy root and the feature selection is executed using at least two selected from the group consisting of RF (Random Forest), PAM (Prediction Analysis for Microarray), and PLS-DA (Partial Least Squares-Discriminant Analysis) to select and analyze a common metabolite, whereby the ages of 4- to 6-year-old ginseng roots can be determined.
